(19) United States
(12) Patent Application Publication
(10) Pub. No.: US 2015/0190377 A1
(43) Pub. Date: Jul. 9, 2015

(54) NEURONAL REGENERATION

(71) Applicants: The Regents of the University of California, a California corporation, Oakland, CA (US); Children's Medical Center Corporation, Boston, MA (US)

(72) Inventors: Vijayendran Chandran, Los Angeles, CA (US); Daniel Geschwind, Santa Monica, CA (US); Giovanni Coppola, Los Angeles, CA (US); Clifford Woolf, Newton, MA (US); Alice Zhang, Los Angeles, CA (US)

(21) Appl. No.: 14/590,712

(22) Filed: Jan. 6, 2015

Related U.S. Application Data
(60) Provisional application No. 61/923,985, filed on Jan. 6, 2014.

(51) Int. Cl. A61K 31/417; A61K 31/137; A61K 31/353

(52) U.S. Cl. CPC A61K 31/417; A61K 31/353; A61K 31/137

(57) ABSTRACT

There are provided, inter alia, methods and compositions useful for neuronal regeneration, including methods for increasing expression of a regeneration-associated marker gene, and methods for increasing neuronal growth.

### CROSS-REFERENCES TO RELATED APPLICATIONS

This application claims the benefit of U.S. Provisional Application No. 61/923,985, filed Jan. 6, 2014, the entire content of which is incorporated herein by reference in its entireties and for all purposes.

### REFERENCE TO A “SEQUENCE LISTING,” A TABLE, OR A COMPUTER PROGRAM LISTING APPENDIX SUBMITTED AS AN ASCII TEXT FILE

The Sequence Listing written in file 83263_{—}928567_ST25.TXT, created on Dec. 31, 2014, 13,810 bytes, machine format IBM-PC, MS-Windows operating system, is hereby incorporated by reference.

### BACKGROUND OF THE INVENTION

The regenerative capacity of injured adult mammalian central nervous system (CNS) is extremely limited, which leads to permanent neurological deficits following CNS injury. In contrast, injured axons in the adult mammalian peripheral nervous system (PNS) maintain the capacity to regenerate, which improves the potential for functional recovery after peripheral nerve injury (Abe et al. 2008). Decades of research have demonstrated that the failure of CNS axon to regenerate is due to many factors, including both low intrinsic regenerative ability (Afshari et al. 2009; Giger et al. 2010; G. M. Smith et al. 2010) and extrinsic inhibitory effects (Filbin 2003; Yiu et al. 2006; Silver et al. 2004; Busch et al. 2007). The importance of intrinsic neuronal signals during injury (Sun et al.) is highlighted by only limited axon regeneration observed after eliminating known extrinsic inhibitory signals (Yiu et al. 2006; Wu et al. 2009).

Currently there are no treatments for spinal cord or for that matter any central nervous system (CNS) injury. There is a limited and incomplete knowledge regarding the core gene network responsible for neuronal regeneration, there are no screens designed to detect small molecules that promote regeneration and there are no drugs approved that enhance CNS regeneration. The present invention addresses these and other needs in the art.

### BRIEF SUMMARY OF THE INVENTION

Provided herein, inter alia, is identification of the core regeneration associated gene network and its transcriptional drivers; several novel genes that enhances neurite outgrowth in DRG neurons; and key transcription factor regulators that are differentially expressed between CNS and PNS after nerve injury. Also provided is an in silico screen using a pattern matching algorithm to compare the expression profiles produced by drugs in cell lines with the core regeneration network. Further provided is identification of an FDA approved drug, ambroxol, from the screen that is capable of significantly enhancing neurite outgrowth in DRG neurons, enhancing CNS regeneration in vivo, and/or repurposing in nerve or CNS repair. Thus, ambroxol has been identified as a useful agent, inter alia, in augmenting PNS or CNS regeneration after injury (repurposing). Additional compounds have been identified as capable of inhibiting PTEN or SCOS3, which in turn increases regeneration after injury.

Accordingly, in a first aspect there is provided a method for increasing neuronal growth in a subject in need thereof. The method includes administering to the subject an effective amount of a compound capable of increasing expression of a regeneration-associated marker gene (RAG).

In another aspect, there is provided a method for increasing neuronal growth in a subject in need thereof. The method includes administering to the subject an effective amount of a compound that inhibits expression or activity of a RAG-repressor, wherein the inhibition of the RAG-repressor increases expression of a regeneration-associated marker gene (RAG).

### BRIEF DESCRIPTION OF THE DRAWINGS

FIGS. 1A-1F. Network analysis of sensory neuron profile changes after sciatic nerve (SN) lesions. FIGS. 1A-1B: Gene dendrograms for two SN lesion datasets. FIGS. 1C-1F: Consensus module preservation across datasets. FIGS. 1C, 1F: Eigengene (first principal component of gene expression) adjacencies of two datasets, rows and columns correspond to one eigengene consensus module. FIG. 1D: Histogram depicting preservation measure for each consensus eigengene. FIG. 1E: Overall module preservation among SN lesion datasets; rows and columns correspond to a consensus module; saturation denotes module preservation.

FIGS. 2A-2D. Experimental validation of novel candidate RAGs. FIGS. 2A-2B: Histograms of differences in neurite outgrowth produced by over-expression of 16 cDNA clones in lentiviral expression vectors with an eGFP expression tag in cultured adult C57BL/6 DRG neurons with Cdc42, as positive control. Total neurite length (FIG. 2A) and number of neurites per neuron (FIG. 2B) quantified using ImageJ software (NeuronJ plugin); from 50-150 cells per view. Significant differences determined by ANOVA with Bonferroni-Holm post hoc test 10/16 candidates induce greater neurite growth. Legend (FIGS. 2A-2B) (histogram bins left to right): Control, Cdc42, Fxyd5, Kif22, RGD130456, Cldn4, Fam46a, Pdcl3, Rrad, Smagp, Gfpt, Rfxap, Nudt6, Grem2, LOC688459, Tslp, Cdc42se2. FIG. 2C: Knock down of the top four selected genes using lentiviral delivery of shRNA with eGFP reporter in C57BL/6J DRGs. Transfected (white arrow) and non-transfected (gray arrow) individual DRG neurons are highlighted. FIG. 2D: Average total neurite length relative control, all data shown is significant relative to control p<0.05. mean±SEM.

FIGS. 3A-3F. Transcription-factor binding-site (TFBS) enrichment in injured regenerating neurons. FIG. 3A: Sequence logo plots of reference (JASPAR) and identified position weight matrix (MEME) for each TF significantly over-represented in magenta module. FIG. 3B: Histogram depicting differential mRNA expression for candidate MZF1 target genes in down-regulated modules by qPCR of DRG neurons over-expressing MZF1 (5 days). Legend (FIG. 3B histogram bins left to right): Gabbr2, Htr3a, Nrg1, Ntrk1, Rgs3, Scn11a, Slcla3, Tnk2, Trpv1, Mzf1_{—}3, Mzf1_{—}2 and Mzf1_{—}1. FIGS. 3C-3D: Histograms depicting change in DRG neurite growth produced by over-expression of MZF1: Total neurite length (FIG. 3C) and longest neurite (FIG. 3D); ANOVA with Bonferroni-Holm post hoc test. FIGS. 3E-3F: Mzf1 knockdown reduces DRG neurite outgrowth. FIG. 3E: Photomicrographs of bIII-tubulinimmunostained DRG neurons transduced with lentivirus non-target control or anti-Mzf1 shRNA at 1 DIV, replated at 8 DIV and cultured for an additional 24 hours for neurite outgrowth. FIG. 3F: Histogram depicting neurite outgrowth after treatment with anti-Mzf1 shRNA mean±S.E. n=173-174 wells, three separate experiments. * p<0.05, Student's two-tailed t test. Scale bar, 250 μm.

FIGS. 4A-4H. Over-represented TFs are involved in transcriptional cross-talk between regeneration-associated pathways. FIG. 4A: Protein-protein interaction network of differentially expressed genes after nerve injury. Nodes correspond to genes and edges to protein-protein interaction (PPI). Larger nodes correspond to number of PubMed hits with co-occurrence of gene and neuronal regeneration, axonal regeneration, nerve injury tags. Node representations: up-regulation, down-regulation, and over-represented TFs. FIG. 4B: PPI network dissociation after in silico removal of over-represented TFs. FIG. 4C: Histogram depicting significantly enriched KEGG pathways (Benjamini corrected P-values<0.05) in the PPI network. FIG. 4D: Histogram depicting distribution of the shortest path between pairs of nodes in the PPI network with or without in silico removal of over-represented TFs. Random removal of similar number of nodes shown for comparison. For each path length (y-axis) of FIG. 4D, the individual bins are in order (top to bottom): Network with TFs; Network without TFS (P-val: 0.006); 9 nodes random removal (P-val: 0.30); and 9 nodes random removal (P-val: 0.49). FIG. 4E-4H: Boxplot representation of the variability in the expression levels of the over-represented TFs between CNS and PNS injury. Time series data after CNS or PNS injury were used to create distance matrix using Euclidean distance measure to create boxplot. Non-parametric Kruskal-Wallis test used to compare differences between CNS and PNS injury datasets. Legend for FIGS. 4E-4H (left to right): CNS injury, PNS injury.

FIGS. 5A-5F. Targeting candidate RAG regulatory network using small-molecules. Gene expression signatures after PNS injury were used to query drug-related expression profiles in the Connectivity Map. Using a pattern-matching algorithm, we selected three drugs (ambroxol, disulfiram and lasalocid) based on enrichment and specificity scores. FIG. 5A: Protein-protein interaction (edges) network of co-expressed and differentially expressed genes (nodes) after PNS injury depicting up-regulation and down-regulation after SN lesion; up-regulation and down-regulation after ambroxol treatment (from Connectivity Map). FIG. 5B: Histogram depicting differences in DRG neurite outgrowth after treatment with drugs. Ambroxol elicited more neuronal growth than control (p<0.05, t-test). FIG. 5C: Histogram depicting differential expression of mRNA for critical genes in PPI network validated by qPCR of DRG neurons treated with 60 mM ambroxol (4 days). Legend (FIG. 5C, top to bottom): ATF3, CASP3, EGR1, FOS, JAK2, JUN, KLF4, MYC, PLAUR, RELA, SMAD1, SP1, STAT1 and STAT3: FIGS. 5D-5F: Ambroxol concentration response. FIG. 5D: Ambroxol produced increases in well fluorescence in a neurite outgrowth assay using DRG neurons from Thy1-YFP reporter mice (one-way ANOVA and Dunnett's post-hoc test (p<0.05, n=10)). FIG. 5E: Photomicrographs of DRG neurons in presence of vehicle (control) or 40 μM ambroxol, immunostained for βIII-tubulin. Images acquired using ImagExpressmicro. Scale bar, 50 μm. FIG. 5F: Histogram depicting concentration-response for neurite outgrowth per neuron vs. ambroxol concentration, mean±SEM for three independent experiments.

FIGS. 6A-6E. Ambroxol promotes retinal ganglion cell axonal regeneration. FIG. 6A: Ambroxol (Amb-25 mg/ml) or vehicle (Veh) was injected into the eye just before optic nerve crush (ONC-Day 1). Animals received daily 120 mL of ambroxol (25 mg/ml) or vehicle by intraperitoneal (IP) injection from day 1 to day 14. At day 7 ambroxol (25 mg/ml) or vehicle was injected into the eye. Tracer CTB was injected into the eye on day 11 and animals sacrificed on day 14. FIG. 6B: Representative retina whole mount images with Tuj1 antibody staining 2 weeks post injury and (FIG. 6C) histogram depicting quantification of retinal ganglion cell (RGCs) survival measured by Tuj1 antibody staining Scale bar: 25 um FIG. 6D: Representative confocal images of optic nerve sections from WT animal treated with vehicle (n=10) and WT animal treated with ambroxol (25 mg/ml, n=13). Axons are labeled with CTB. Scale bar: 100 um. Measurements were made blinded to treatment. FIG. 6F: Quantification of number of axons in FIG. 6D as function of distance from crush site. T-test ** p<0.001 * p<0.05.

FIGS. 7A-7D. Candidate RAGs regulate neurite outgrowth regulatory pathways. Quantitative real-time RT-PCR and western blot assessment of RAP markers after over-expression of candidate RAGs. Stable PC12 neuronal cell lines individually over-expressing full-length mouse ORF cDNA clones of four candidate RAGs (Fxyd5, Gfpt1, Smagp and Tacstd2) were utilized for all the analyses. FIG. 7A: Network display of quantitative real-time RT-PCR data is shown, where nodes represent experimental condition (candidate RAG overexpression) and RAP marker genes, and the number on the edges represents the fold change when compared relative to the controls. FIG. 7B: RAGs' effects on NOG converge on several signaling pathways. Regeneration associated pathways along with their corresponding marker genes tested (genes in rectangle), showing up-regulated pathway marker genes (denoted in rectangle) during the over-expression of RAGs (denoted in oval). FIG. 7C: Western blot analyses for regeneration associated marker proteins after overexpression of candidate RAGs. FIG. 7D: Histograms depicting relative time course gene expression levels of four novel candidate RAGs after SN lesion in rats DRGs (left to right: Fxyd4, Smagp, Gfpt1 and Tacstd2). Legend of times (left to right): Day 0, Day 1, Day 3, Day 7 and Day 14.

FIG. 8. Nuerite growth and expansion with respect to RAGs. FIG. 8 histogram depicts average number of neurites under described experimental conditions. Histogram legend (left to right): Control, Cdc42, Fxyd5, Tacstd2, Kif22, RGD1304563, Cldn4, Fam46a, Pdcl3, Rrad, Smagp, Gfpt1, Rfxap, Nudt5, Grem2, LOC688459, Tslp and Cdc42se2.

FIG. 9. Schematic depiction of protein interaction network of the over-represented TFs.

FIG. 10. Quantitative real-time RT-PCR after treatment of candidate drugs. N2A cell lines were treated with the corresponding drugs for 48 hours and the expression levels of the marker genes was measured by q-RT-PCR, using Gapdh as internal control. Nodes represents the genes tested, the edges represent the direction of regulation (i.e., up-regulation, downregulation) and the edge size represents the extent of fold change.

FIG. 11. Correlations between coexpression module eigengenes and the nerve injury process. In each cell the Pearson correlation coefficient is shown with the corresponding p-value in brackets following. Cells in the table are coded using correlation values according to scale on the right.

FIGS. 12A-12E. Histograms depicting correlation values comparing the direction of correlation based on the gene expression levels of the top 50 hub genes in each identified modules in 15 (7 PNS and 8 CNS) independent datasets related to PNS and CNS neuronal injury. Lower triangle of the correlation matrix generated from pairwise PNS versus PNS and PNS versus CNS datasets were utilized to generate these histogram plots. Legend: FIG. 12A (magenta module); FIG. 12B (Pink module); FIG. 12C (Purple module); FIG. 12D (Darkred module); FIG. 12E (Greenyellow module). Within each of FIGS. 12A-12E, histogram bins are set left to right at −1 to −0.5, −0.5 to 0, 0 to 0.5, and 0.5 to 1. Within each bin of FIGS. 12A-12E, the entries appear in the order PNS (left) and CNS (right). FIGS. 12A,12B depict up-regulated modules. FIGS. 12C, 12D and 12E depict down-regulated modules.

FIGS. 13A-13D. Western blot analysis of cultured human (293) (FIG. 13A), mouse (N2A) (FIG. 13B) and rat (PC12) (FIG. 13C) cells treated with Luteolin (P2) and Quercetin (P6). FIG. 13D: Protein levels of PTEN, AKT, pAKT (Ser473) and beta-Tubulin were measured. Increased concentration of Luteolin (P2) and Quercetin (P6) showed increased expression level of phosphorylated form of AKT denoting PTEN inhibition.

FIG. 14. Further use of gene expression data to identify drugs to enhance regeneration. FIG. 14 is a schematic flowchart depicting integrated various of data sets disclosed herein to find core set of 32 drugs. Remarkably, these compounds largely fall into 4 classes, a highly significant convergent result signifying that we have identified core structure—activity relationships that will promote neural regeneration.

FIGS. 15A-15E. Ambroxol promotes retinal ganglion cell axonal regeneration in PTEN knockout mice. Ambroxol (Amb-25 mg/ml) or vehicle (Veh) was injected into the eye just before optic nerve crush. Animals received daily 300 mg/kg of ambroxol or vehicle by intraperitoneal (IP) injection for the first five days after the crush and then they received 150 mg/kg until day 14. At day 7 ambroxol (25 mg/ml) or vehicle was injected into the eye. Tracer CTB was injected into the eye on day 11 and animals sacrificed on day 14. FIG. 15A: Representative confocal images of optic nerve sections from PTEN^{-/-} animal treated with vehicle (n=4) and PTEN^{-/-} animal treated with ambroxol (n=4). Axons are labeled with CTB. Scale bar: 100 um. Measurements were made blinded to treatment. FIG. 15B: Quantification of number of axons in FIG. 7A. T-test ** p<0.01 * p<0.05. FIG. 15C: Representative retina whole mount images with Tuj1 and P-S6 antibody staining 2 weeks post injury and FIGS. 15D-15E: Quantification of retinal ganglion cell (RGCs) survival measured by Tuj1 and P-S6 antibody staining Scale bar: 25 um.

### DETAILED DESCRIPTION OF THE INVENTION

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

Thus, the compounds of the present invention may exist as salts, such as with pharmaceutically acceptable acids. The present invention includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. Unless expressly indicated to the contrary, as used herein the terms “patient” and “subject” are synonymous.

An “effective amount” is an amount sufficient to accomplish a stated purpose (e.g. achieve the effect for which it is administered, treat a disease, reduce enzyme activity, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, PHARMACEUTICAL DOSAGE FORMS (vols. 1-3, 1992); Lloyd, THE ART, SCIENCE AND TECHNOLOGY OF PHARMACEUTICAL COMPOUNDING (1999); Pickar, DOSAGE CALCULATIONS (1999); and REMINGTON: THE SCIENCE AND PRACTICE OF PHARMACY, 20th Edition, 2003, Gennaro, Ed., Lippincott, Williams & Wilkins).

The term “contacting” may include allowing two species to react, interact, or physically touch.

As defined herein, the term “inhibition”, “inhibit”, “inhibiting” and the like means negatively affecting (e.g. decreasing) an activity or function (e.g. of an enzyme or biological process) relative to the activity or function in the absence of the inhibitor.

The term “modulator” refers to a composition that increases or decreases the level of a target molecule or the function of a target.

“Patient” or “subject in need thereof” refers to a living organism suffering from or prone to a condition that can be treated by administration of a pharmaceutical composition as provided herein. Non-limiting examples include humans, other mammals, bovines, rats, mice, dogs, monkeys, goat, sheep, cows, deer, and other non-mammalian animals. In embodiments, a patient is human. In embodiments, a patient is bovine. In embodiments, a patient is a cow. In embodiments, a patient is mammal.

“Disease” or “condition” refer to a state of being or health status of a patient or subject capable of being treated with the compounds or methods provided herein

As used herein, the term “neurodegenerative disease” refers to a disease or condition in which the function of a subject's nervous system becomes impaired. Examples of neurodegenerative diseases that may be treated with a compound or method described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, frontotemporal dementia, Gerstmann-Straussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoffs disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, or Tabes dorsalis.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine. and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

Methods.

In one aspect there is provided a method for increasing neuronal growth in a subject in need thereof. The method includes administering to the subject an effective amount of a compound capable of increasing expression of a regeneration-associated marker gene (RAG). The increasing of neuronal growth is an increase relative to the amount of neuronal growth in the absence of the compound.

The compound may increase expression of at least one or more RAGs (relative to the amount of expression in the absence of the compound). The increased expression may result from a compound that increases the activity of a RAG (e.g. RAG-enhancing compound). The increased expression may result from inhibiting a RAG-repressor (e.g. a gene or protein in a RAG signaling pathway capable of repressing RAG activity). In embodiments, the RAG-repressor is PTEN or SOCS3. The RAG-repressor may be PTEN. The RAG-repressor may be SOCS3. Thus, compounds herein, including embodiments thereof, may inhibit the RAG-repressor thereby increasing expression of one or more RAGs.

In embodiments, the RAG is Fxyd5, Gfpt1, Smagp, Tacstd2, Kif22, RGD1304563, Cldn4, Fam46a, Rfxap or Pdcl3. In embodiments, the RAG is Fxyd5. In embodiments, the RAG is Gfpt1. In embodiments, the RAG is Gfpt1. In embodiments, the RAG is Smagp. In embodiments, the RAG is Tacstd2. In embodiments, the RAG is Kif22. In embodiments, the RAG is RGD1304563. In embodiments, the RAG is Cldn4. In embodiments, the RAG is Fam46a. In embodiments, the RAG is Rfxap. In embodiments, the RAG is Pdcl3.

In embodiments, the compound is capable of increasing expression of one, two, three, four, five, six, seven, eight, nine or all of Fxyd5, Gfpt1, Smagp, Tacstd2, Kif22, RGD1304563, Cldn4, Fam46a, Rfxap and Pdcl3.

In embodiments, the RAG is Fxys5, Gfpt1, Smagp or Tacstd2. In embodiments, the compound is capable of increasing expression of one, two, three or all four of Fxys5, Gfpt1, Smagp and Tacstd2 (relative to the amount of expression in the absence of the compound).

In embodiments, the compound is capable of increasing the activity of a transcription factor selected from the group consisting of ATF3, CREB1, CTCF, EGR1, FOS, FOXI1, JUN, KLF4, MZF1, NFATC2, NFIL3, NFKB1, RARA, REL, RELA, REST, RORA, SMAD1, SOX11, SP1, STAT1, STAT3, and TFAP2A (relative to the amount of activity in the absence of the compound). In embodiments, the compound is capable of increasing the activity of one, two, three, four, five, six, seven, eight, nine or all of ATF3, CREB1, CTCF, EGR1, FOS, FOXI1, JUN, KLF4, MZF1, NFATC2, NFIL3, NFKB1, RARA, REL, RELA, REST, RORA, SMAD1, SOX11, SP1, STAT1, STAT3, and TFAP2A.

In embodiments, the compound is a Na+ channel blocker or a Ca2+ channel blocker. In embodiments, the compound is a Na+ channel blocker. In embodiments, the compound is a Ca2+ channel blocker. In embodiments, the compound suppresses symptoms of neuropathic pain. In embodiments, the compound suppresses symptoms of neuropathic spinal cord injury.

The compound may activate a RAG. In embodiments, the compound is ambroxol (e.g., trans-4-(2-Amino-3,5-dibrombenzylamino)-cyclohexanol) or a derivative thereof (as known in the art, including for example amborxol hydrochloride and N-acylated ambroxol derivatives TEI-588a, TEI-588b, TEI-589a, TEI-589b, TEI-602a (aromatic amine-acylated derivative) and TEI-602b (aliphatic amine-acylated derivative)). See e.g. Biochem Biophys Res Commun. 2009 Mar. 13; 380(3):586-90. Epub 2009 Jan. 25. Action of N-acylated ambroxol derivatives on secretion of chloride ions in human airway epithelia. Yamada T, Takemura Y, Niisato N, Mitsuyama E, Iwasaki Y, Marunaka Y, which is hereby incorporated by reference for all purposes. In embodiments, the compound is ambroxol. In embodiments, the compound is an ambroxol derivative, the derivative having one or more chemical substitutions relative to ambroxol. The compound may be provided in a pharmaceutical composition including a pharmaceutically acceptable excipient. In embodiments, the pharmaceutical composition includes pharmaceutically acceptable salts of the compound. In embodiments, the compound is covalently attached to a carrier moiety. In embodiments, the compound is non-covalently linked to a carrier moiety. In embodiments, the compound is a pharmaceutically acceptable salt of ambroxol. In embodiments, the compound is a pharmaceutically acceptable salt of an ambroxol derivative.

In embodiments, the compound inhibits a RAG-repressor, thereby increasing expression of the RAG. In embodiments, the -repressor is PTEN or SOCS3. In embodiments, the repressor is PTEN. In embodiments, the repressor is SOCS3. In embodiments, the compound is luteolin, quercetin, genistein, or phentolamine. In embodiments, the compound is luteolin. In embodiments, the compound is quercetin. In embodiments, the compound is genistein. In embodiments, the compound is phentolamine.

The compound may inhibit activity of PTEN (e.g. a PTEN-inhibitor) or inhibit the activity of SOCS3 (e.g. a SOCS3-inhibitor). The compound may thus be a PTEN-inhibitor. In embodiments, the PTEN-inhibitor is a compound as set forth in Table 1 following. In embodiments, the PTEN-inhibitor is luteolin or quercetin. The PTEN inhibitor may be luteolin. The PTEN inhibitor may be quercetin. The compound may be a SOCS3-inhibitor. In embodiments, the SOCS3-inhibitor is a compound as set forth in Table 1. In embodiments, the SOCS3-inhibitor is genistein or phentolamine. The SOCS3-inhibitor may be genistein. The SOCS3-inhibitor may be phentolamine. The RAG-repressor inhibitor compound may be provided in a pharmaceutical composition including a pharmaceutically acceptable excipient. In embodiments, the pharmaceutical compositions include pharmaceutically acceptable salts of the compound. In embodiments, the compound is covalently attached to a carrier moiety. In embodiments, the compound is non-covalently linked to a carrier moiety.

TABLE 1
Structures and Embodiments of Compounds Described Herein.

| Drug-ID | Name | Structure | Target |
|---|---|---|---|
| Abx | ambroxol (C₁₃H₁₉Br₂ClN₂O) | 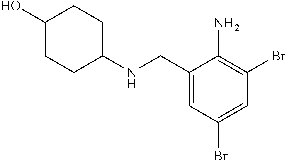 [SMILES: Nc1c(Br)cc(Br)cc1CNC1CCC(O)CC1] | Br Core PNS network |
| P2 | luteolin (C₁₅H₁₀O₆) | 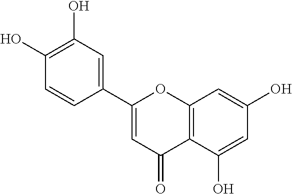 [SMILES: O=c1cc(-c2ccc(O)c(O)c2)oc2cc(O)cc(O)c12] | PTEN inhibition |
| P6 | quercetin (C₁₅H₁₀O₇) | 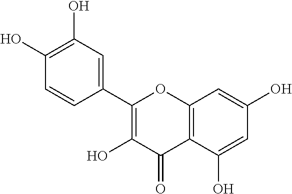 [SMILES: O=c1c(O)c(-c2ccc(O)c(O)c2)oc2cc(O)cc(O)c12] | PTEN inhibition |
| S5 | genistein (C₁₅H₁₀O₅) | 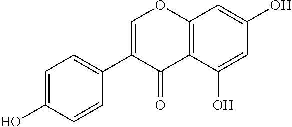 [SMILES: O=c1c(-c2ccc(O)cc2)coc2cc(O)cc(O)c12] | SOCS3 inhibition |
| S8 | phentolamine (C₁₇H₁₉N₃O) | 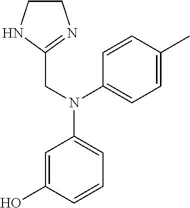 [SMILES: Cc1ccc(N(CC2=NCCN2)c2cccc(O)c2)cc1] | SOCS3 inhibition |
| | felodipine | 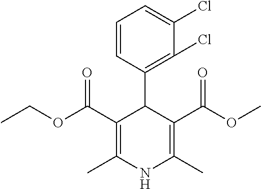 [SMILES: CCOC(=O)C1=C(C)NC(C)=C(C(=O)OC)C1c1cccc(Cl)c1Cl] | L-type Ca channel blocker; Also potential mTOR activity |
| | ticlopidine | 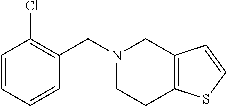 [SMILES: Clc1ccccc1CN1CCc2sccc2C1] | |
| | sulconazole | 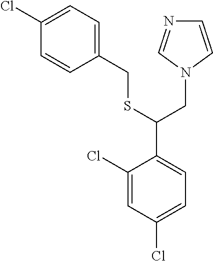 [SMILES: Clc1ccc(CSC(Cn2ccnc2)c2ccc(Cl)cc2Cl)cc1] | Anti-fungal-azole |
| | propofol | 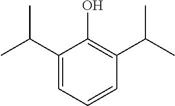 | Anesthetic; GABA channel blocker; sodium channel blocker |
| | isoconazole | 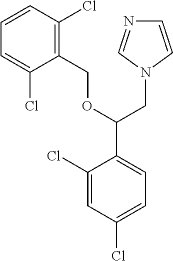 [SMILES: Clc1ccc(C(Cn2ccnc2)OCc2c(Cl)cccc2Cl)c(Cl)c1] | Anti-fungal azole |
| | azacyclonol | 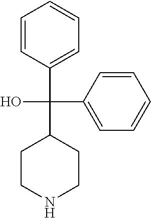 [SMILES: OC(c1ccccc1)(c1ccccc1)C1CCNCC1] | |
| | prenylamine | 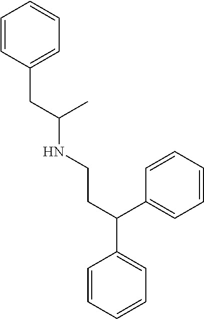 [SMILES: CC(Cc1ccccc1)NCCC(c1ccccc1)c1ccccc1] | Depletes myocardial catecholamine stores and has some calcium channel blocking activity |
| | fendiline | 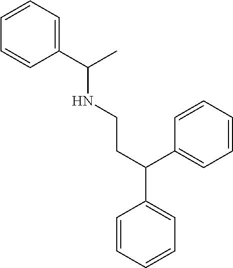 [SMILES: CC(NCCC(c1ccccc1)c1ccccc1)c1ccccc1] | Coronary vasodilator; inhibits calcium function in muscle cells in excitation- contraction coupling; proposed as antiarrhythmic and antianginal agents. |
| | hexetidine | 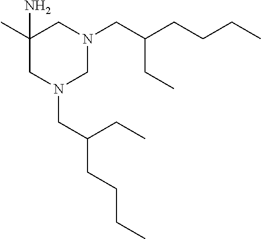 [SMILES: CCCCC(CC)CN1CN(CC(CC)CCCC)CC(C)(N)C1] | A bactericidal and fungicidal antiseptic. It is used as a 0.1% mouthwash for local infections and oral hygiene. |
| | cloperastine | 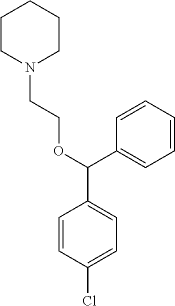 [SMILES: Clc1ccc(C(OCCN2CCCCC2)c2ccccc2)cc1] | |
| | drofenine | 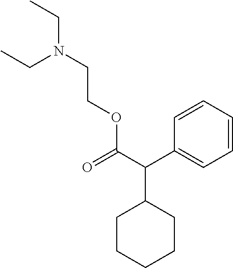 [SMILES: CCN(CC)CCOC(=O)C(c1ccccc1)C1CCCCC1] | |
| | felodipine | 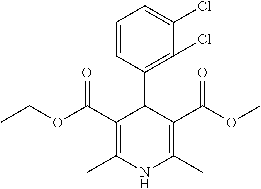 [SMILES: CCOC(=O)C1=C(C)NC(C)=C(C(=O)OC)C1c1cccc(Cl)c1Cl] | A dihydropyridine calcium antagonist with positive inotropic effects. It lowers blood pressure by reducing peripheral vascular resistance through a highly selective action on smooth muscle in arteriolar resistance vessels. |
| | dienestrol | 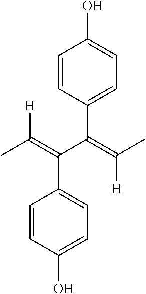 [SMILES: C/C=C(/C(=C/C)c1ccc(O)cc1)c1ccc(O)cc1] | A synthetic, non-steroidal estrogen structurally related to stilbestrol. It is used, usually as the cream, in the treatment of menopausal and postmenopausal symptoms. |
| | clioquinol | 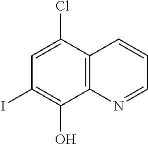 [SMILES: Oc1c(I)cc(Cl)c2cccnc12] | A potentially neurotoxic 8- hydroxyquinoline derivative long used as a topical anti-infective, intestinal antiamebic, and vaginal trichomonacide. The oral preparation has been shown to cause subacute myelo-optic neuropathy and has been banned worldwide. |
| | ivermectin | 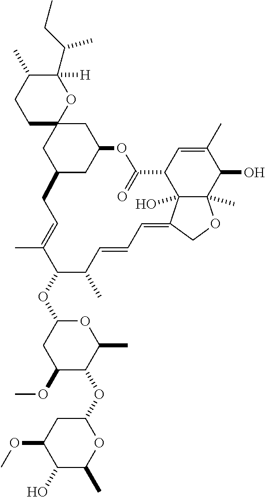 | It binds glutamate-gated chloride channel to cause increased permeability and hyperpolarization of nerve and muscle cells. It also interacts with other chloride channels. It is a broad spectrum antiparasitic that is active against microfilariae of Onchocerca volvulus but not the adult form. |
| | clorgiline | 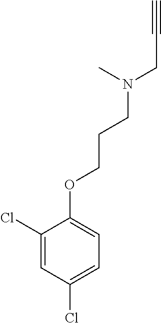 [SMILES: C#CCN(C)CCCOc1ccc(Cl)cc1Cl] | An antidepressive agent and monoamine oxidase inhibitor related to oargyline. |
| | naftifine | 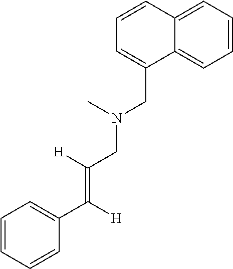 [SMILES: CN(C/C=C/c1ccccc1)Cc1cccc2ccccc12] | Naftifine is a synthetic, broad spectrum, antifungal agent and allylamine derivative for the topical treatment of tinea pedis, tinea cruris, and tinea corporis caused by the organisms Trichophyton rubrum, Trichophyton mentagrophytes, Trichophyton tonsurans and Epidermophyton floccosum. |
| | quinisocaine | 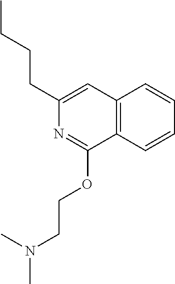 [SMILES: CCCCc1cc2ccccc2c(OCCN(C)C)n1] | |
| | mefloquine | 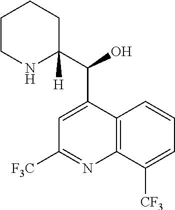 [SMILES: O[C@@H](c1cc(C(F)(F)F)nc2c(C(F)(F)F)cccc12)[C@H]1CCCCN1] | A phospholipid-interacting antimalarial drug (antimalarials). It is very effective against Plasmodium falciparum with very few side effects. |
| | miconazole | 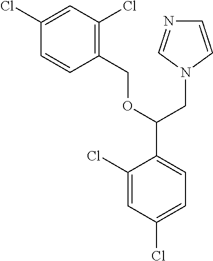 [SMILES: Clc1ccc(COC(Cn2ccnc2)c2ccc(Cl)cc2Cl)c(Cl)c1] | An imidazole antifungal agent that is used topically and by intravenous infusion. |
| | clomifene | 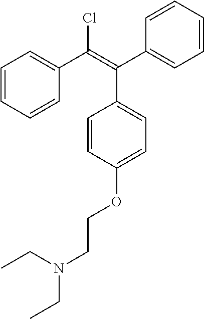 [SMILES: CCN(CC)CCOc1ccc(/C(=C(/Cl)c2ccccc2)c2ccccc2)cc1] | A triphenyl ethylene stilbene derivative which is an estrogen agonist or antagonist depending on the target tissue. |
| | oxybutynin | 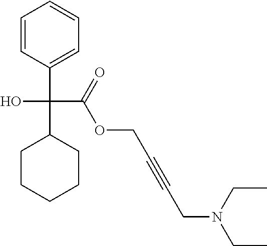 [SMILES: CCN(CC)CC#CCOC(=O)C(O)(c1ccccc1)C1CCCCC1] | Oxybutynin is an anticholinergic medication used to relieve urinary and bladder difficulties, including frequent urination and inability to control urination, by decreasing muscle spasms of the bladder. It competitively antagonizes the M1, M2, and M3 subtypes of the muscarinic acetylcholine receptor. |
| | loperamide | 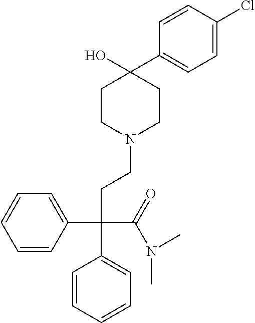 [SMILES: CN(C)C(=O)C(CCN1CCC(O)(c2ccc(Cl)cc2)CC1)(c1ccccc1)c1ccccc1] | One of the long-acting synthetic antidiarrheals; it is not significantly absorbed from the gut, and has no effect on the adrenergic system or central nervous system, but may antagonize histamine and interfere with acetylcholine release locally. |
| | butoconazole | 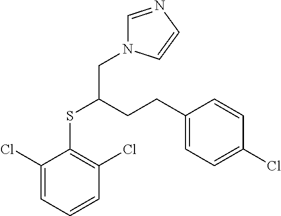 [SMILES: Clc1ccc(CCC(Cn2ccnc2)Sc2c(Cl)cccc2Cl)cc1] | Butoconazole is an imidazole antifungal used in gynecology. |
| | profenamine | 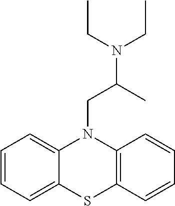 [SMILES: CCN(CC)C(C)CN1c2ccccc2Sc2ccccc21] | A medication derived from phenothiazine. It is primarily used as an antidyskinetic to treat parkinsonism. |
| | vanoxerine | 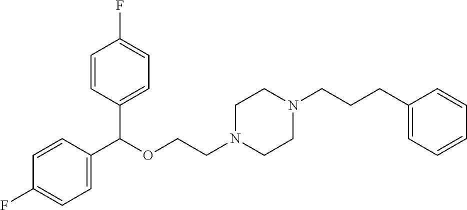 [SMILES: Fc1ccc(C(OCCN2CCN(CCCc3ccccc3)CC2)c2ccc(F)cc2)cc1] | |
| | famprofazone | 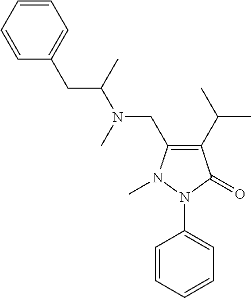 [SMILES: CC(C)c1c(CN(C)C(C)Cc2ccccc2)n(C)n(-c2ccccc2)c1=O] | |
| | chlorhexidine | 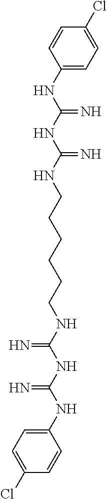 [SMILES: N=C(NCCCCCCNC(=N)NC(=N)Nc1ccc(Cl)cc1)NC(=N)Nc1ccc(Cl)cc1] | A disinfectant and topical anti- infective agent used also as mouthwash to prevent oral plaque. |
| | bromperidol | 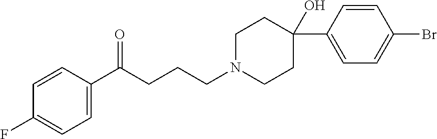 [SMILES: O=C(CCCN1CCC(O)(c2ccc(Br)cc2)CC1)c1ccc(F)cc1] | |
| | amoxapine | 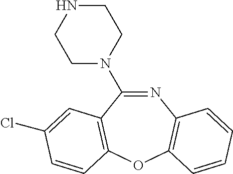 [SMILES: Clc1ccc2c(c1)C(N1CCNCC1)=Nc1ccccc1O2] | The N-demethylated derivative of the antipsychotic agent loxapine that works by blocking the reuptake of norepinephrine, serotonin, or both. It also blocks dopamine receptors. |

In another aspect, there is provided a method for increasing neuronal growth in a subject in need thereof. The method includes administering to the subject an effective amount of a compound that inhibits expression or activity of a RAG-repressor, wherein the inhibition of the RAG-repressor increases expression of a regeneration-associated marker gene (RAG). The increasing of neuronal growth is relative to the amount of neuronal growth in the absence of the compound. Thus, the compound may inhibit activity of a RAG-repressor (e.g. a RAG-repressor inhibitor). The inhibition of the RAG-repressor may increase RAG expression. In embodiments, the RAG-repressor inhibitor compound is a compound as set forth in Table 1.

In embodiments, the compound is ambroxol, luteolin, quercetin, genistein, phentolamine, felodipine, ticlodipine, sulconazole, propofol, isoconazole, azacyclonol, prenylamine, fendiline, hexetidine, cloperastine, drofenine, dienestrol, clioquinol, ivermectin, clorgiline, naftifine, quinisocaine, mefloquine, miconazole, clomifene, oxybutynin, loperamide, butoconazole, profenamine, vanoxerine, famprofazone, chlorhexidine, bromperidol, or amoxapine.

In embodiments, the RAG-repressor inhibitor is ambroxol, luteolin, quercetin, genistein, or phentlamine. In embodiments, the RAG-repressor inhibitor is luteolin, quercetin, genistein, phentolamine. In embodiments, the RAG-repressor inhibitor is ambroxol. In embodiments, the RAG-repressor inhibitor is luteolin. In embodiments, the RAG-repressor inhibitor is quercetin. In embodiments, the RAG-repressor inhibitor is genistein. In embodiments, the RAG-repressor inhibitor is phentolamine.

In embodiments, the compound inhibits a RAG-repressor, thereby increasing expression of the RAG (relative to the amount of expression in the absence of the compound). In embodiments, the -repressor is PTEN or SOCS3. In embodiments, the repressor is PTEN. In embodiments, the repressor is SOCS3. In embodiments, the compound is luteolin, quercetin, genistein, or phentolamine. In embodiments, the compound is luteolin. In embodiments, the compound is quercetin. In embodiments, the compound is genistein. In embodiments, the compound is phentolamine.

Further to any method disclosed herein, in embodiments, the neuronal growth is neuronal regeneration. In embodiments, the neuronal regeneration includes accelerating or improving neural repair in the CNS of the subject. In embodiments, the subject has experienced a traumatic injury to the CNS. In embodiments, the neuronal regeneration includes accelerating or improving neural repair in the PNS of the subject. In embodiments, the neuronal regeneration includes restoring neuronal function in the subject. In embodiments, the subject has a neurodegenerative disease. Thus, in embodiments, provided herein is a method of treating a neurological disease in a subject (e.g., a human subject) in need thereof. The method includes administering a therapeutically effective amount of a compound capable of increasing expression of a regeneration-associated marker gene (RAG) and/or a compound that inhibits expression or activity of a RAG-repressor, wherein the inhibition of the RAG-repressor increases expression of a regeneration-associated marker gene (RAG), as disclosed herein. In embodiments, the compound is capable of increasing expression of a regeneration-associated marker gene (RAG), as disclosed herein. In embodiments, the compound inhibits expression or activity of a RAG-repressor, wherein the inhibition of the RAG-repressor increases expression of a regeneration-associated marker gene (RAG), as disclosed herein.

Pharmaceutical Compositions.

In another aspect, there is provided a pharmaceutical composition including a pharmaceutically acceptable excipient in combination with a compound capable of increasing expression of a RAG, or in combination with a compound that inhibits expression or activity of a RAG-repressor, wherein the inhibition of the RAG-repressor increases expression of a RAG.

The pharmaceutical compositions include optical isomers, diastereomers, or pharmaceutically acceptable salts of the modulators disclosed herein. The compound included in the pharmaceutical composition may be covalently attached to a carrier moiety, as described above. Alternatively, the compound included in the pharmaceutical composition is not covalently linked to a carrier moiety.

The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g. to reduce metabolic degradation).

In embodiments, one or more compounds that activate RAGs may be co-administered with RAG-repressor inhibitors as described herein, including embodiments thereof. In embodiments, one or more compounds that activate RAGs may co administered. In embodiments, one or more RAG-repressor inhibitors may be co administered.

The compounds of the present invention can be prepared and administered in a wide variety of oral, parenteral and topical dosage forms. Oral preparations include tablets, pills, powder, dragees, capsules, liquids, lozenges, cachets, gels, syrups, slurries, suspensions, etc., suitable for ingestion by the patient. The compounds of the present invention can also be administered by injection, that is, intravenously, intramuscularly, intracutaneously, subcutaneously, intraduodenally, or intraperitoneally. Also, the compounds described herein can be administered by inhalation, for example, intranasally. Additionally, the compounds of the present invention can be administered transdermally. It is also envisioned that multiple routes of administration (e.g., intramuscular, oral, transdermal) can be used to administer the compounds of the invention. Accordingly, the present invention also provides pharmaceutical compositions comprising a pharmaceutically acceptable excipient and one or more compounds of the invention.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substance, that may also act as diluents, flavoring agents, binders, preservatives, tablet disintegrating agents, or an encapsulating material.

In powders, the carrier is a finely divided solid in a mixture with the finely divided active component (e.g. a compound provided herein. In tablets, the active component is mixed with the carrier having the necessary binding properties in suitable proportions and compacted in the shape and size desired. The powders and tablets preferably contain from 5% to 70% of the active compound.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, for example, push-fit capsules made of gelatin, as well as soft, sealed capsules made of gelatin and a coating such as glycerol or sorbitol.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

When parenteral application is needed or desired, particularly suitable admixtures for the compounds of the invention are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampules are convenient unit dosages. The compounds of the invention can also be incorporated into liposomes or administered via transdermal pumps or patches. Pharmaceutical admixtures suitable for use in the present invention are well-known to those of skill in the art and are described, for example, in Pharmaceutical Sciences (17th Ed., Mack Pub. Co., Easton, Pa.) and WO 96/05309, the teachings of both of which are hereby incorporated by reference.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg, more typically 1.0 mg to 1000 mg, most typically 10 mg to 500 mg, according to the particular application and the potency of the active component. The composition can, if desired, also contain other compatible therapeutic agents.

Some compounds may have limited solubility in water and therefore may require a surfactant or other appropriate co-solvent in the composition. Such co-solvents include: Polysorbate 20, 60 and 80; Pluronic F-68, F-84 and P-103; cyclodextrin; polyoxyl 35 castor oil; or other agents known to those skilled in the art. Such co-solvents are typically employed at a level between about 0.01% and about 2% by weight.

Viscosity greater than that of simple aqueous solutions may be desirable to decrease variability in dispensing the formulations, to decrease physical separation of components of a suspension or emulsion of formulation and/or otherwise to improve the formulation. Such viscosity building agents include, for example, polyvinyl alcohol, polyvinyl pyrrolidone, methyl cellulose, hydroxypropyl methylcellulose, hydroxyethyl cellulose, carboxymethyl cellulose, hydroxy propyl cellulose, chondroitin sulfate and salts thereof, hyaluronic acid and salts thereof, combinations of the foregoing, and other agents known to those skilled in the art. Such agents are typically employed at a level between about 0.01% and about 2% by weight. Determination of acceptable amounts of any of the above adjuvants is readily ascertained by one skilled in the art.

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

Pharmaceutical compositions provided by the present invention include compositions wherein the active ingredient is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., modulating the activity of a target molecule (e.g. prion protein, amyloid beta, alpha-synuclein, huntingtin), and/or reducing, eliminating, or slowing the progression of disease symptoms. Determination of a therapeutically effective amount of a compound of the invention is well within the capabilities of those skilled in the art, especially in light of the detailed disclosure herein.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated (e.g., prion disease, protein misfolding disease, Creutzfeldt-Jakob disease, Gerstmann-Sträussler-Scheinker syndrome, kuru), kind of concurrent treatment, complications from the disease being treated or other health-related problems. Other therapeutic regimens or agents can be used in conjunction with the methods and compounds of Applicants' invention. Adjustment and manipulation of established dosages (e.g., frequency and duration) are well within the ability of those skilled in the art.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present invention should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. In one embodiment, the dosage range is 0.001% to 10% w/v. In another embodiment, the dosage range is 0.1% to 5% w/v.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

The ratio between toxicity and therapeutic effect for a particular compound is its therapeutic index and can be expressed as the ratio between LD₅₀ (the amount of compound lethal in 50% of the population) and ED₅₀ (the amount of compound effective in 50% of the population). Compounds that exhibit high therapeutic indices are preferred. Therapeutic index data obtained from cell culture assays and/or animal studies can be used in formulating a range of dosages for use in humans. The dosage of such compounds preferably lies within a range of plasma concentrations that include the ED₅₀ with little or no toxicity. The dosage may vary within this range depending upon the dosage form employed and the route of administration utilized. See, e.g. Fingl et al., In: THE PHARMACOLOGICAL BASIS OF THERAPEUTICS, Ch.1, pal, 1975. The exact formulation, route of administration and dosage can be chosen by the individual physician in view of the patient's condition and the particular method in which the compound is used.

The compositions of the present invention can be delivered by transdermally, by a topical route, formulated as applicator sticks, solutions, suspensions, emulsions, gels, creams, ointments, pastes, jellies, paints, powders, and aerosols.

The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

The pharmaceutical compositions of the present invention can be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use.

In another embodiment, the compositions of the present invention are useful for parenteral administration, such as intravenous (IV) administration or administration into a body cavity or lumen of an organ. The formulations for administration will commonly comprise a solution of the compositions of the present invention dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present invention in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In another embodiment, the formulations of the compositions of the present invention can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The compounds described herein can be used in combination with one another, with other active agents known to be useful in treating a disease associated with misfolded proteins, prion proteins, or protein aggregates, or with adjunctive agents that may not be effective alone, but may contribute to the efficacy of the active agent.

In embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

In embodiments, a pharmaceutical composition as described herein includes a compound selected from any of the tables, figures, or charts provided herein.

### EXAMPLES

### Example 1

### Identifying Intrinsic Molecular Factors

Identifying the various intrinsic molecular factors/signals controlling regeneration of axons in PNS injury models would significantly advance our understanding of neuronal regenerative mechanism and pathways accounting for the differences in the regenerative capacity of PNS and CNS neurons, permitting us to identify the key differences that underlie the non-regenerative state of the CNS and potentially accelerate PNS recovery as well. Several over-expression and knockout studies of candidate genes has provided evidence for minimal but not complete recovery after CNS injury (Kevin Kyungsuk Park et al. 2008; P. D. Smith et al. 2009; Sun et al. 2011; Liu et al. 2010), demonstrating the presences of differences in neuronal growth states between PNS and CNS injury. Elucidating the in vivo molecular state during PNS injury and recovery in recapitulating this molecular state in the CNS after injury would be an effective approach to better understand CNS injury recovery.

One of the intrinsic molecular mechanisms contributing to the regenerative process is the retrograde transport of injury signals to the cell body of the neuron, leading to the expression of regeneration-associated genes (RAGs). Rather than studying individual genes, the differences between the quiescent PNS and the regenerating PNS neuron, and the contrast of these processes with CNS reflect major differences in transcriptional states, reflecting regenerative and growth potential. Thus a multi-staged systems biology approach was applied to characterize the transcriptional network associated with neurite outgrowth in PNS. Changes in gene expression responsible for increasing the intrinsic growth state after PNS injury, but that do not typically occur in the CNS, where identified to pinpoint the transcriptional changes sufficient for promoting increased regenerative capacity in the CNS.

The repair of traumatic injuries to the central nervous system (CNS) sufficient to restore function presents a significant therapeutic challenge. Current therapies to treat brain and spinal cord injuries are deficient. The regenerative capacity of the injured adult mammalian CNS is extremely limited, which leads to permanent neurological deficits. The compounds and methods discovered inter alia, provide for treatment of injuries. Deletion of PTEN (phosphatase and tensin homolog), a negative regulator of the mammalian target of rapamycin (mTOR) pathway, and/or SOCS3 (suppressor of cytokine signaling 3), a negative regulator of Jak/STAT pathways, in adult retinal ganglion cells promotes robust, and long distance, axon regeneration after optic nerve injury (Park et al., 2008; Smith et al., 2009; Sun et al., 2011). Importantly, similar genetic manipulation also led to robust regeneration of injured corticospinal tract (CST) axons after different spinal cord injury models (Sun et al., 2011). However, these models are based on gene deletion and do not exploit expression of the newly defined core transcriptional profile to screen for small molecule drugs that can promote regeneration, including a repurposing of existing drugs, such as ambroxol, as discovered herein. Examples disclosed herein employed ambroxol as trans-4-(2-Amino-3,5-dibrombenzylamino)-cyclohexanol.

Data sets used for initial computational meta-analysis, obtained from various nerve injury models, are tabulated in Table 2 following.

TABLE 2
Data sets employed herein.

| Dataset | No. of | | | |
|---|---|---|---|---|
| No. | Arrays | Experiment | Time | Reference |
| 1 | 16 | DRG (L4,5,6) after SN lesion | 0, 3, 7, 14 Days | [17] |
| | | (vs.naive) | | |
| 2 | 25 | DRG after C3 lesion (vs. naive) | 0, 3, 7, 14 Days | [18] |
| 3 | 43 | DRG after SN lesion (vs. sham) | 1, 3, 8, 12, 16, 18, 24, 28 hrs | [19] |
| 4 | 21 | DRG after spinal nerve ligation | 0, 3, 7, 21, 40 Days | p20[ |
| | | (vs.sham) | | |
| 5 | 21 | DRG after spared nerve injury | 0, 3, 7, 21, 40 Days | [20] |
| | | (vs.sham) | | |
| 6 | 21 | DRG after chronic nerve | 0, 3, 7, 21, 40 Days | [20] |
| | | constriction (vs. sham) | | |
| | Total: | | Time points: 31 | |
| | 147 | | | |

Construction of RAG Co-Expression Networks.

Neuropathy occurs as a consequence of a variety of injuries to both the CNS and PNS. We began by analyzing the transcriptional changes following nerve injury at multiple time points ranging from hours to days after injury from several validated experimental models of nerve injury (Beggs et al. 2006): SN lesion (sciatic nerve transection), C3 lesion (Cervical cord hemisection at the C3 level), spinal nerve ligation, spared nerve injury, and chronic nerve constriction generated in 3 different laboratories (in total 31 time points).

The systems biology approach taken to elucidate the nerve injury related transcriptome structure by performing weighted gene co-expression network analysis (WGCNA) (Langfelder et al. 2008; Geschwind et al. 2009) on time series data gathered after SN lesion performed in 2 different laboratories. This allows for independently reproducible networks.

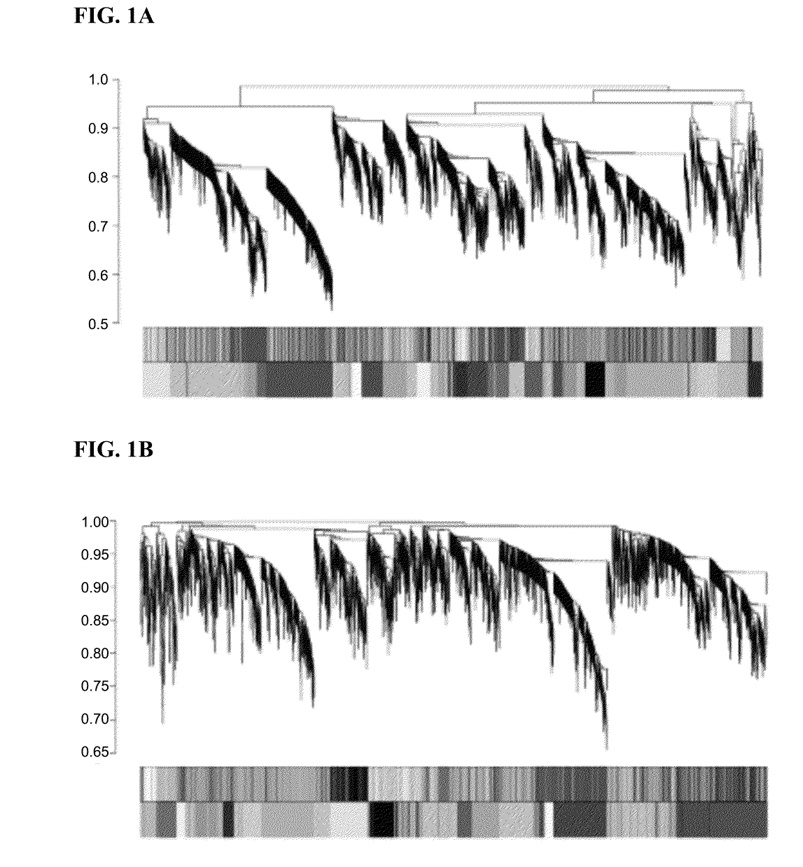

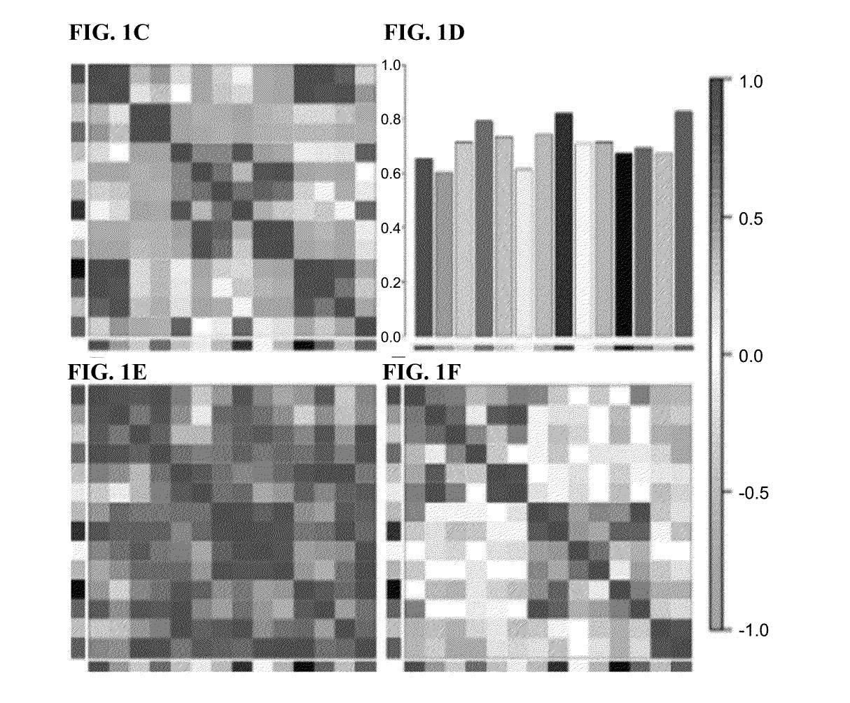

Independent WGCNA was performed on these two time series, which permits the identification of modules of highly co-expressed genes related to specific functional pathways (Langfelder et al. 2008; Oldham et al. 2008; Konopka et al. 2009; Wexler et al. 2011), and the key hub genes, or drivers, within the major outgrowth related module or modules, which would serve as candidate RAGs for further experimental validation. Identification of fourteen co-expression modules (FIGS. 1A-1F), and based on their eigengene adjacency to time-dependent changes after injury, allowed classification as up-regulated, down-regulated and early-regulated co-expression modules. Additional analysis revealed up and down-regulated regeneration-associated co-expression modules; two up-regulated after nerve injury (magenta [394 genes] and pink [74 genes]) and three down-regulated modules (purple [194 genes], darkred [52 genes], and greenyellow [53 genes]) after injury (FIGS. 1A-1B).

Consensus module analysis identified conserved modules shared between the two SN lesion datasets from the connectivity patterns or module structures (Langfelder et al. 2007, 2008). This network represents the intersection of the co-expression networks from these two independent experiments, providing a robust depiction of the modular structure (Langfelder et al. 2007, 2008). This analysis indicates that the gene co-expression relationships identified following SN injury are highly preserved, which represent biological pathways that are shared among the two datasets; these fourteen modules represent underlying common pathways and genes involved in regeneration after nerve injury (FIGS. 1C-1F). These co-expression based modules provide a key tool with which to explore the relationships among genes expression that govern the biological function in question (Barabasi et al. 2004), in this case neuronal regeneration. Each module's association was examined with neuronal regeneration based on published literature by testing association with the key-words neuronal-regeneration, axonal-regeneration, and nerve injury in the PubMed database for every gene. This analysis identified that the magenta module is significantly enriched for genes associated with neuronal regeneration (hypergeometric P-value 3.4×10⁻¹¹). Nearly 22% of genes (n=88 out of 394 genes in module) were previously shown to be associated in neural regeneration or axon outgrowth.

Validation of RAG Co-Expression Modules.

The direction of gene expression of the top 50 hub genes, which represent the most central genes in the co-expression network, was compared in 15 (7 PNS and 8 CNS) independent datasets related to either PNS or CNS neuronal injury. Preservation of the correlation relationships was observed in the PNS injury data with these entirely independently generated PNS injury data sets (r2 range, 0.5 to 1.0). Furthermore, a higher degree (>1.5 fold) of anti-correlation (≦−0.5) for gene expression levels in the CNS compared to the PNS datasets was observed. This analysis validates that these PNS injury related transcriptional networks are robust and that they are not preserved in the CNS. Furthermore, to validate these co-expression modules and identify robust pathways and RAGs involved in neuronal regeneration after injury, the direction of gene expression in up and down-regulated modules was examined in 13 independent datasets related to neuronal injury. This analysis revealed consistency in the changes in gene expression levels in all the datasets analyzed when compared with the nerve injury datasets, providing validation of these transcriptional networks related to neuronal regeneration after injury. To validate and identify gene clusters associated with common functional categories, we next applied Gene Ontology (GO) enrichment analyses which would suggest the functional importance of these co-expressed genes after injury. This showed enrichment (Benjamini corrected P-values<0.05) for several GO categories in the up-regulated RAG co-expression modules (magenta and pink) that are functionally associated with neuronal regeneration (the significant clusters were: regulation of transcription, neuron differentiation, inflammation, stimulus related, signaling related, and cell proliferation/growth/migration). GO functional analysis for down-regulated RAG co-expression modules (purple and darkred) revealed enrichment for various categories related to plasma membrane, ion/gated channel, ion binding and synapse/cell junction.

Table 3 following discloses a Gene Ontology analysis of nerve injury associated modules. For categorization and clustering of GO terms, we considered GO terms with Benjamini corrected P-values less than 0.05. The total number of genes present in each module is represented within brackets.

TABLE 3
Gene Ontology analysis

| | | No. of | No. of |
|---|---|---|---|
| Module | GO annotation clusters | GO terms | Genes |
| Magenta (530) | Cluster 1: Regulation of transcription related | 16 | 70 |
| Up-regulated | Cluster 2: Stimulus related | 12 | 56 |
| | Cluster 3: Inflammation/wounding related | 7 | 48 |
| | Cluster 4: Apoptosis related | 9 | 38 |
| | Cluster 5: Signaling related | 2 | 29 |
| | Cluster 6: Cell proliferation/growth related | 4 | 25 |
| | Cluster 7: Neuron differentiation | 1 | 22 |
| | Cluster 8: Cell migration related | 5 | 13 |
| Pink (131) | Cluster 1: Extracellular matrix/region related | 7 | 22 |
| Up-regulated | | | |
| Purple (281) | Cluster 1: Plasma membrane related | 2 | 55 |
| Down-regulated | Cluster 2: Ion/gated channel activity related | 17 | 32 |
| | Cluster 3: Ion binding related | 2 | 26 |
| | Cluster 4: Synapse/cell junction related | 4 | 17 |
| Darkred (57) | Cluster 1: Ion binding related | 2 | 18 |
| Down-regulated | Cluster 2: Ion/gated channel activity related | 10 | 15 |
| Greenyellow (112) | Not significant | N/A | N/A |
| Down-regulated | | | |

Experimental Validation of Candidate RAGs.

To provide experimental validations of the network analysis predictions, genes were selected from the unsupervised co-expression analysis based on their connectivity score within modules and required that they have not been previously associated with neuronal regeneration in the literature: Smagp (small trans-membrane and glycosylated protein), Gfpt1 (glutamine fructose-6-phosphate transaminase 1), Tslp (thymic stromal lymphopoietin), Nudt6 (nucleoside diphosphate linked moiety X-type motif 6), Cdc42se2 (CDC42 small effector 2), Rfxap (regulatory factor X-associated protein), Grem2 (gremlin 2), and LOC688459. All of the above genes were selected from magenta module (up-regulated), except for Gfpt1, which is the hub gene in the pink module (up-regulated). We augmented this validation set utilizing a knowledge-based semi-supervised approach to include the following additional genes with strong co-expression relationships in our datasets to neuronal regeneration: Fxyd5 (FXYD domain containing ion transport regulator 5), Tacstd2 (tumor-associated calcium signal transducer 2), Kif22 (kinesin family member 22), RGD1304563, Cldn4 (claudin 4), Fam46a (family with sequence similarity 46, member A), Pdcl3 (phosducin-like 3), and Rrad (Ras-related associated with diabetes).

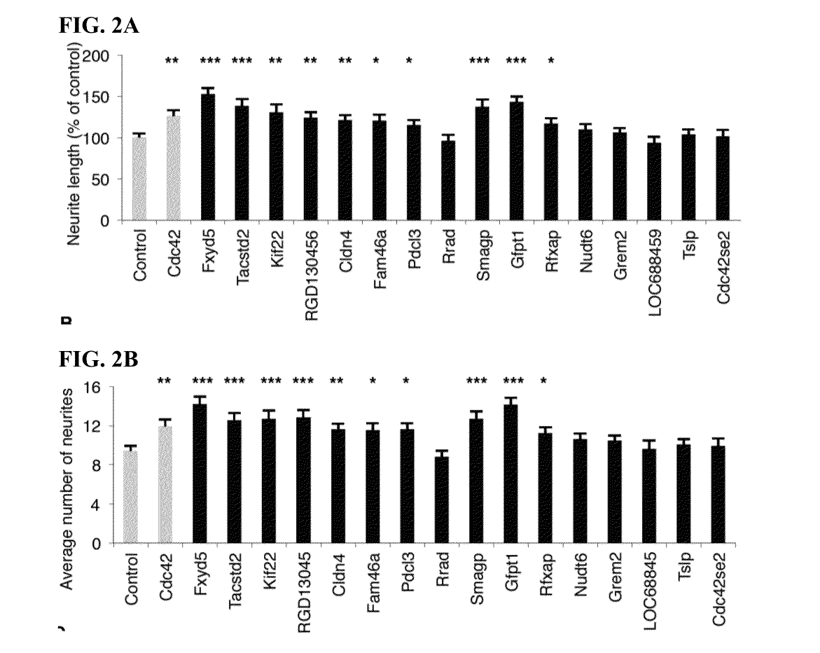

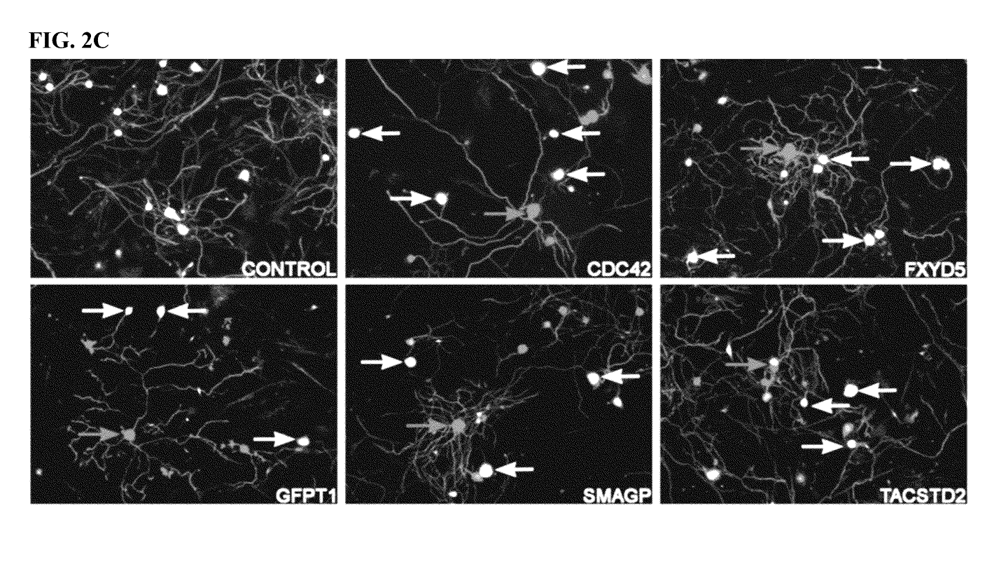

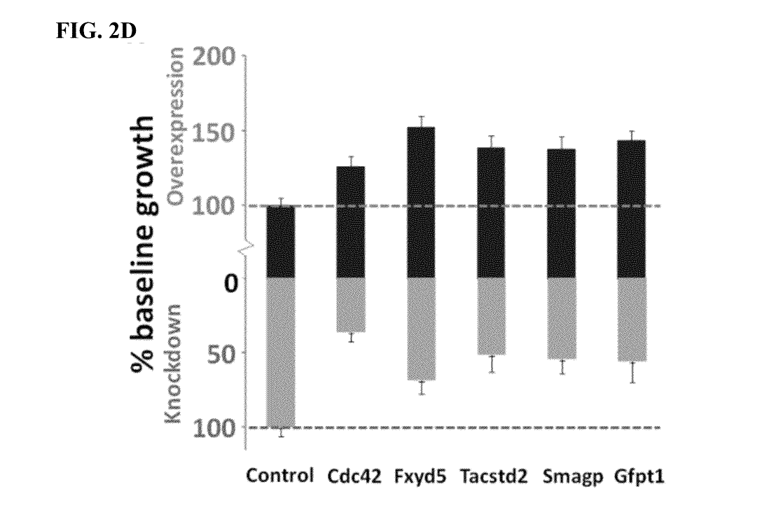

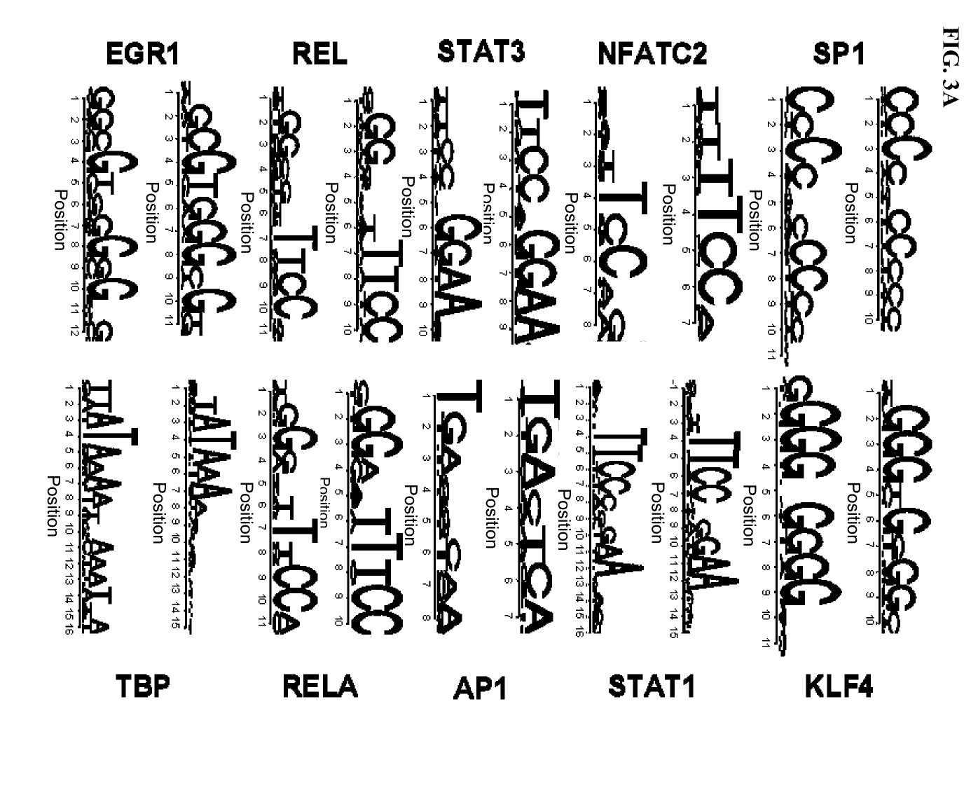

An in vitro assay was performed monitoring neurite outgrowth following over-expression of full-length mouse ORF cDNA clones for each candidate gene in adult mouse DRG neurons. Of the 16 RAGs tested, 10 candidate RAGs exhibited statistically significant increases in neurite length and the number of neurites after over-expression (ANOVA with Bonferroni-Holm post hoc test; FIGS. 2A, 2C, and FIG. 9). None of these genes have previously been reported to be associated with neurite outgrowth or neuronal regeneration (e.g. Fxyd5, Gfpt1, Smagp, Tacstd2, Kif22, RGD1304563, Cldn4, Fam46a, Rfxap and Pdcl3).

Fxyd5 is an ion transport regulator which is involved in the up-regulation of chemokine production (Nam et al. 2007). Gfpt1 is a rate-limiting enzyme of the hexosamine pathway which catalyzes the formation of glucosamine 6-phosphate. Smagp is not a well-studied small trans-membrane glycoprotein known to be altered in cancer (Tarbé et al. 2004). Tacstd2 encodes a carcinoma-associated antigen which acts as cell surface receptor that transduces calcium signals and activates ERK/MAPK signaling pathway in tumor cell lines (Cubas et al. 2010). None of these genes has been previously associated with neuronal regeneration in the literature. RT-PCR and Western blots were performed to see if the over-expression of these candidate RAGs leads to the increased expression of marker genes and proteins associated with pathways involved in neuronal regeneration. The over-expression of these candidate RAGs activates several distinct pathways involved in neuronal regeneration process.

In vitro assays were performed monitoring neurite outgrowth following knock down of candidate RAGs. RNA interference was applied using lentiviral plasmids containing either shRNA sequences to Fxyd5, Gfpt1, Smagp, Tactd2 and Cdc42 or a shRNA control vector (containing a non-specific shRNA) in adult mouse DRG neurons. In direct contrast to the effect of their over-expression, we found in all cases that target knockdown significantly (p<0.05) reduced neurite outgrowth in adult DRG mouse neurons (FIGS. 2B,2C), further validating them as novel genes involved in promoting neurite outgrowth.

Table 4 following sets forth PNS and CNS nerve injury related datasets used for validation. The table summarizes fifteen independent nerve injury related datasets used for validation analyses. 15 (7 PNS and 8 CNS) independent datasets related to PNS and CNS neuronal injury were studied to examine the consistency of the co-expression networks. Microarray data sets were downloaded from the Gene Expression Omnibus.

TABLE 4
PNS and CNS nerve injury related datasets

| | No. of | Experiment | | | |
|---|---|---|---|---|---|
| No. | Arrays | Type | Experiment | GEO Id | Reference |
| 1 | 22 samples | Peripheral | Spared Nerve Injury. Adult rat L4 | GSE30691 | (21, 22) |
| | (9 control, | nerve injury | and L5 DRGs cells after 3, 7, 21, 40 | | |
| | 12 injured) | | hours. | | |
| 2 | 72 samples | Peripheral | Sciatic nerve lesion. Adult rat L4 | GSE26350 | 19) |
| | (21 sham, | nerve injury | and L5 DRGs cells after | | |
| | 51 injured) | | 1, 3, 8, 12, 16, 18, 24, and 28 hours after | | |
| | | | a sciatic nerve (proximal and distal) | | |
| | | | lesion. | | |
| 3 | 12 samples | Peripheral | Chronic Constriction Injury. Adult | GSE30691 | (21, 22) |
| | (12 injured) | nerve injury | rat L4 and L5 DRGs cells after | | |
| | | | 3, 7, 21, 40 hours. | | |
| 4 | 22 samples | Peripheral | Spinal Nerve Ligation. Adult rat L4 | GSE30691 | (21, 22) |
| | (9 sham, 12 | nerve injury | and L5 DRGs cells after 3, 7, 21, 40 | | |
| | injured) | | hours. | | |
| 5 | 36 samples | Peripheral | DRGs from L4 and L5 spinal nerve | GSE2884 | N/A |
| | (12 sham, | nerve injury | ligation model of neuropathic pain | | |
| | 24 inured) | | in the rat (at 28 and 50 days) | | |
| 6 | 18 samples | Peripheral | Proximal sciatic nerve (SN) tissues | GSE33175 | (23) |
| | (0 to 9 | nerve injury | (0.5 cm) at 0 h, 0.5 h, 1 h, 3 h, 6 h and | | |
| | hours | | 9 h after sciatic nerve resection. | | |
| | injured) | | | | |
| 7 | 24 samples | Peripheral | Sciatic nerve crush at 12, 24, 72 | GSE21007 | (24) |
| | (24 injured) | nerve injury | hours and 7 days. Lumbar DRGs | | |
| | | | L4, L5 and L6. | | |
| 8 | 15 samples | Motor | Motor cortex injury | Unpublished | N/A |
| | (15 injured) | cortex | | | |
| | | injury | | | |
| 9 | 5 samples | Optic nerve | Retinal ganglion cells (RGCs) after | N/A | (25) |
| | (5injured) | injury | 4 d post lens injury - with lens injury | | |
| 10 | 5 samples | Optic nerve | Retinal ganglion cells (RGCs) after | N/A | (25) |
| | (5 injured) | injury | 4 d post lens injury - without lens | | |
| | | | injury | | |
| 11 | 5 samples | Optic nerve | Retinal ganglion cells (RGCs) after | N/A | (25) |
| | (5 control) | injury | 4 d post lens injury | | |
| | | (control) | | | |
| 12 | 41 (19 | Spinal cord | Mild spinal cord injury at thoracic | GDS63 | (26) |
| | control, 22 | injury | vertebrae T9 at various time points | | |
| | mild) | | up to 28 days post injury. | | |
| 13 | 29 (29 | Spinal cord | Moderate spinal cord injury at | GDS63 | (26) |
| | moderate) | injury | thoracic vertebrae T9 at various time | | |
| | | | points up to 28 days post injury. | | |
| 14 | 19 (19 | Spinal cord | Severe spinal cord injury at thoracic | GDS63 | (26) |
| | severe) | injury | vertebrae T9 at various time points | | |
| | | | up to 28 days post injury. | | |
| 15 | 31 samples | Spinal cord | Gene expression changes were | GSE19701 | (27) |
| | (31 injured) | transection | studied in rat tail motor neurons 0, | | |
| | | | 2, 7, 21 and 60 days after complete | | |
| | | | spinal transection. | | |

Transcription Factor Binding Site Enrichment in RAG Co-Expression Modules.

Transcription factor binding site (TFBS) enrichment analysis was performed in each of the RAG co-expression modules. For TFBS enrichment analysis, the promoter regions 1000 bp upstream of the transcription start site for all the genes present in a given co-expression module, were analyzed utilizing experimentally defined TFBS position weight matrices (PWMs) from the JASPAR database (Portales-Casamar et al. 2010) to examine the enrichment for corresponding TFBS within each module. Screening promoter sequences of co-expressed genes in a medium window range of 1000 bp would identify core enriched TFBS and eliminate the possibility of false positives corresponding to large noise components (motifs) in DNA sequences affecting the prediction rate (Methods). Three different background datasets (1000 bp sequences upstream of all rat genes, rat CpG islands and the rat chromosome 20 sequence) were used avoid confounders and identify the most statistically robust sites. This analysis identified 18 TFs significantly enriched in RAG co-expression modules (p-value<0.05 relative to all the three background dataset). The up-regulated modules, magenta and pink, showed enrichment for 9 TFs that were previously known to be involved in the neuronal injury response. For example, SP1 (Kiryu-Seo et al. 2008), KLF4 (Moore et al. 2009), FOS, c-Jun (Raivich et al. 2004), STAT3 (Qiu et al. 2005), RELA (Yoon et al. 2008), and ERG1 (Herdegen et al. 1993) were over-represented in the up-regulated module (magenta). Interestingly five of these over-represented TFs were hubs in the magenta module in the up-regulated module after injury: RELA, FOS, EGR1, JUN and STAT3. The TFBS enrichment analysis was cross validated on the sequences upstream of the orthologous genes in mouse and human after combining species-specific up and down-regulated modules separately. 10 of 15 TFs that were over-represented in rats were also over-represented in mouse or humans (SP1, KLF4, AP1, STAT3, STAT1, EGR1, NFATC2, REST, RELA, and MZF1) demonstrating phylogenetic conservation of TFBS in the promoter regions of these co-expressed genes after nerve injury. The enriched TFs related to up-regulated modules (SP1 (Kiryu-Seo et al. 2008), KLF4 (Moore et al. 2009), FOS, c-Jun (Raivich et al. 2004), STAT3 (Qiu et al. 2005), RELA (Yoon et al. 2008), and ERG1 (Herdegen et al. 1993)) are well studied during the nerve injury process when compared to the TFs enriched in the down-regulated modules (candidate transcriptional repressors).

### MZF1, a Novel TF Regulating Process Outgrowth

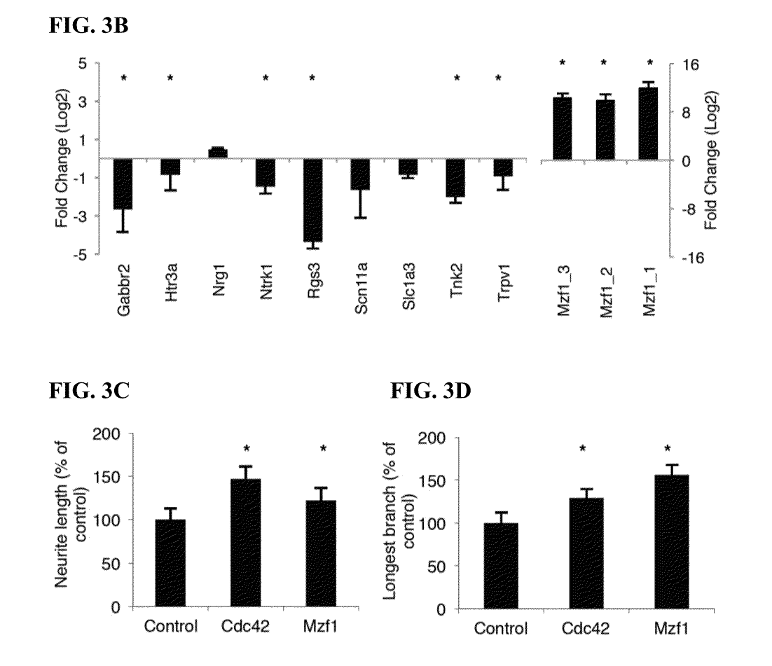

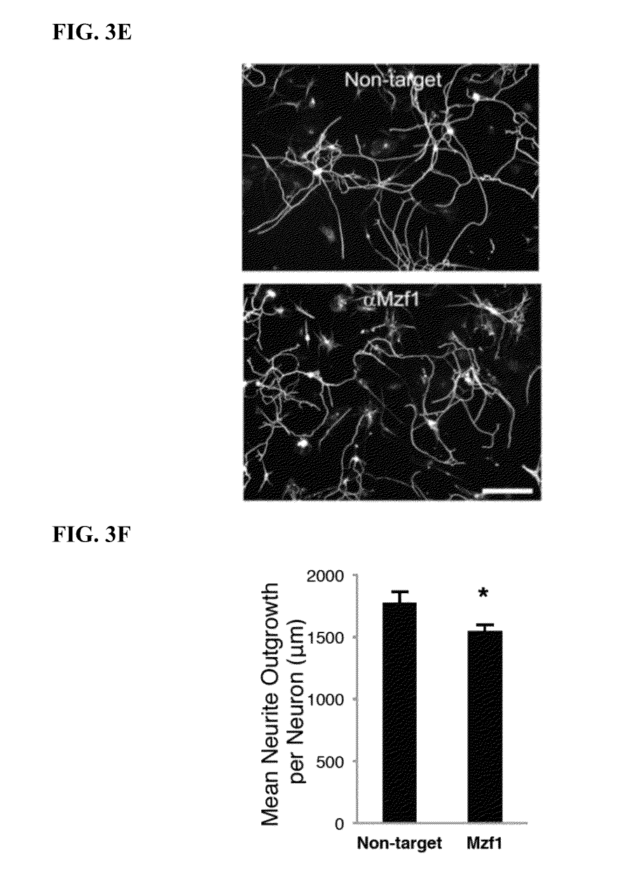

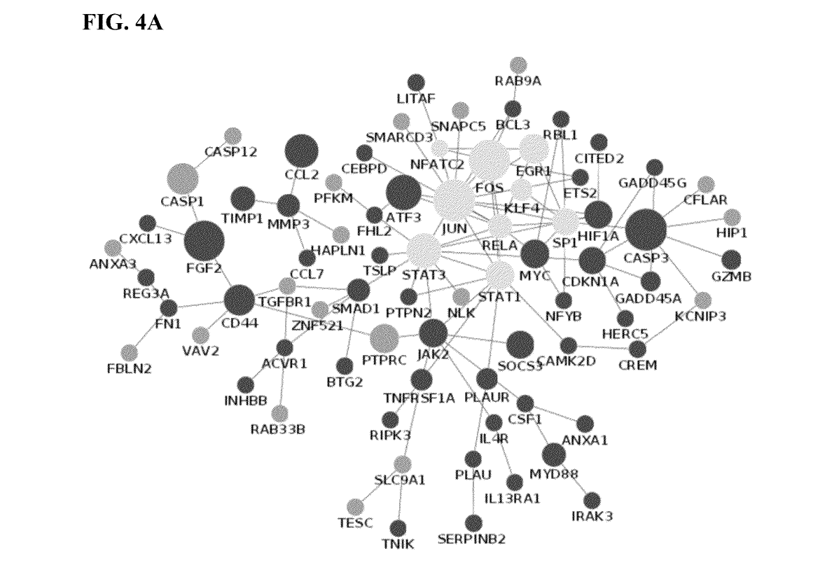

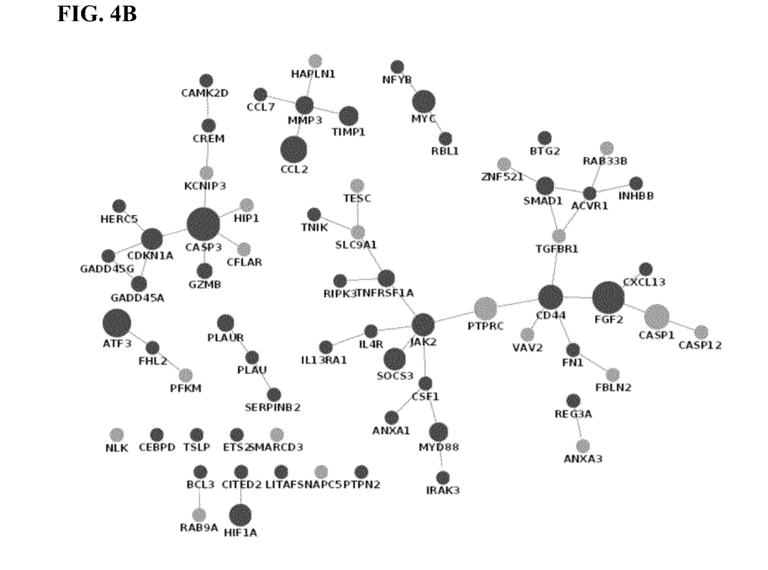

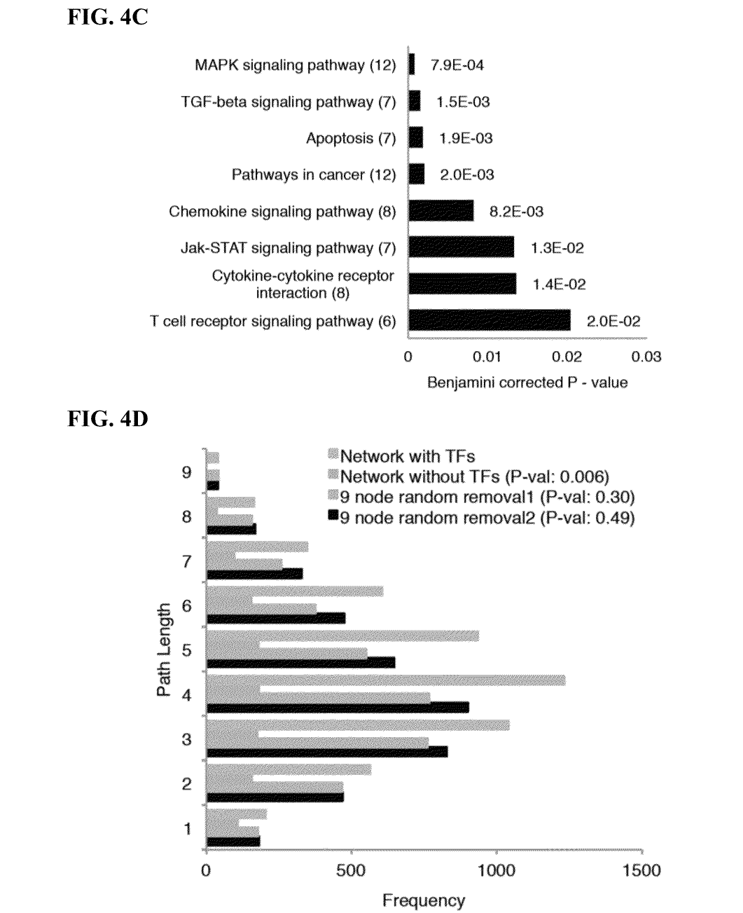

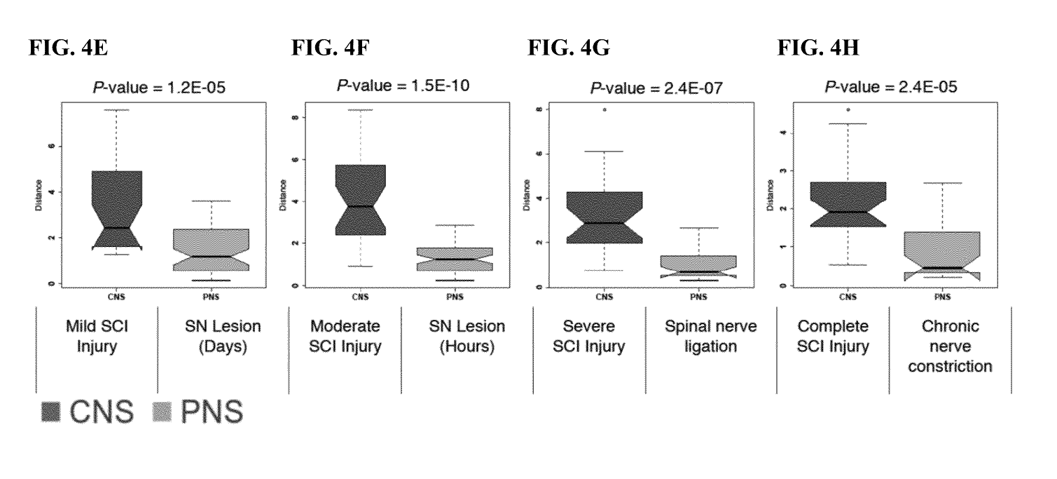

It has been shown that during regeneration program many genes related to ion channels are dynamically regulated, where their coordinated regulation is essential for PNS axonal regeneration (Shim et al. 2010). However, this coordinated dynamic regulation of ion channels after injury is not completely understood (Shim et al. 2010). To specifically address this question, TFs were screened for over-represented in down-regulated modules—purple, darkred and greenyellow, which are enriched in genes related to the GO terms plasma membrane, ion/gated channel, ion binding and synapse/cell junction related. The TF MZF1 (myeloid zinc finger 1) was over-represented in the down-regulated modules, suggesting a potential role in regulation of neurite outgrowth and repair following injury. MZF1 has no previously known role in neurite outgrowth or neuronal regeneration. We over-expressed MZF1 in mouse DRG neurons and examined gene expression of putative targets, validating its role in regulating these specific transcripts. MZF1 was found herein to be over-expressed and produced a significant increase in average neurite length (FIG. 3D) and longest neurite (FIG. 3E) compared with control neurons while MZF1 knockdown significantly (p<0.05) reduced neurite outgrowth in adult DRG neurons (FIGS. 3F-3G). This indicates that TFs that act as repressors to suppress gene expression after nerve injury can increase intrinsic growth and was unknown prior to this work. This implies that constitutive expression of some genes may act as a brake on regenerative capacity. These observations demonstrate that MZF1 is a novel transcription factor that promotes neurite outgrowth, as predicted by the network and bioinformatic analysis.

Co-Regulated Genes Represent Convergent Pathways.

The protein-protein interaction (PPI) network represented by the genes in all the modules was determined. Protein-protein interactions (PPIs) provide important clues for therapeutic intervention targets after nerve injury (Barabasi et al. 2011). Experimental validated PPIs were screened among all possible combinations of gene pairs present in the co-expressed modules and over-represented TFs, obtaining a PPI network consisting of 77 nodes and 102 edges. Despite the relatively small number of proteins in this network, there is enrichment of several pathways that previously have been associated with neuronal regeneration in this PPI network. These include the MAPK signaling pathway, the TGF-beta signaling pathway, the chemokine signaling pathway, and the Jak-STAT signaling pathway (Abe et al. 2008). Indeed, several TFs over-represented in the RAG co-expression modules were hubs in this network and many genes belonging to these enriched signaling pathways were also enriched for the TFBS of the over-represented TFs, consistent with this observation.

This suggested that these core regeneration associated TF provided key regulatory mechanisms and connecting these distinct signaling pathways related to neural process outgrowth. These nine TFs from the PPI network were tested, and the resulting network mean path length examined, which is a measure of the connectivity of the remaining protein interactions in the absence of these TF. This analysis demonstrated a drastic and significant reduction in protein connectivity (from 87 to 23% of connected pairs, p=0.0024), strongly indicating that these TFs provide important influences over the signaling cross-talk mechanisms between the enriched pathways responsible for neuronal regeneration.

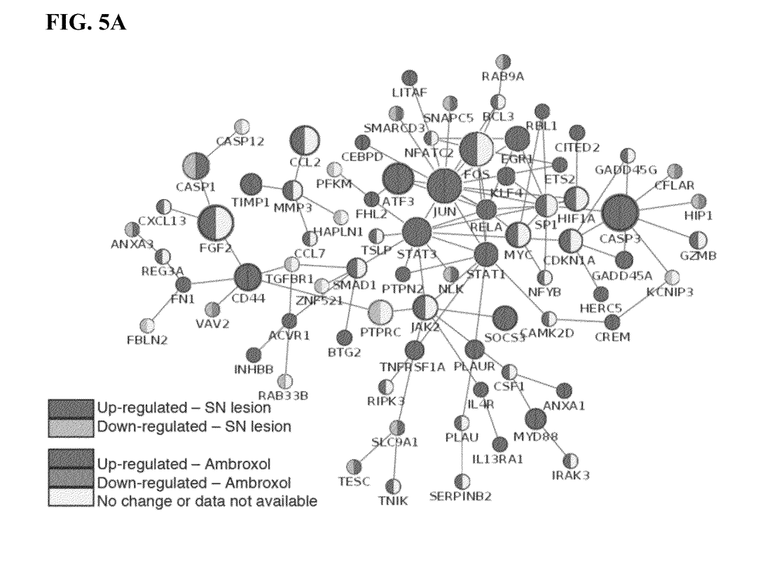

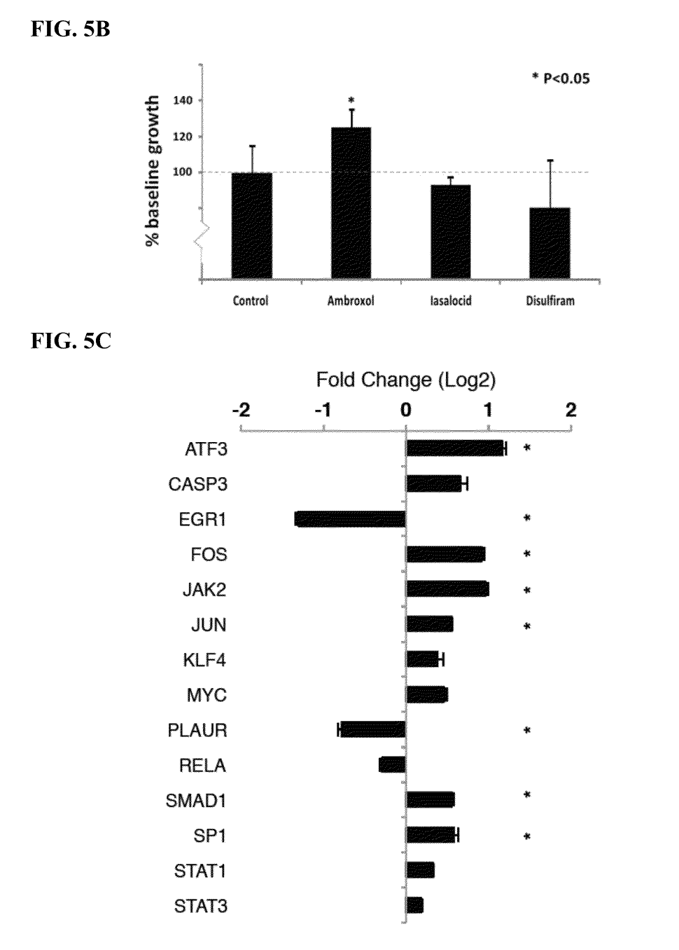

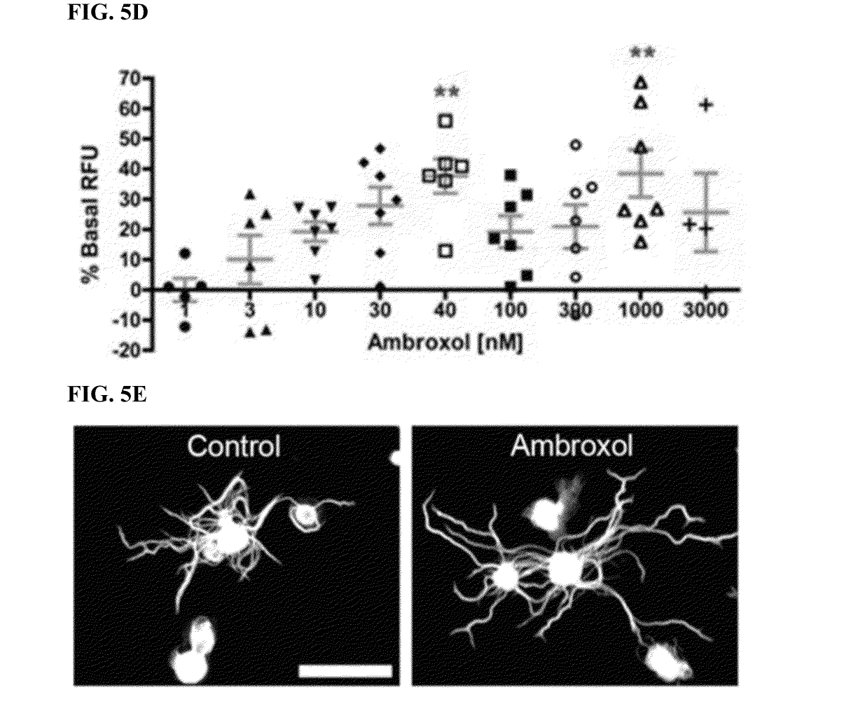

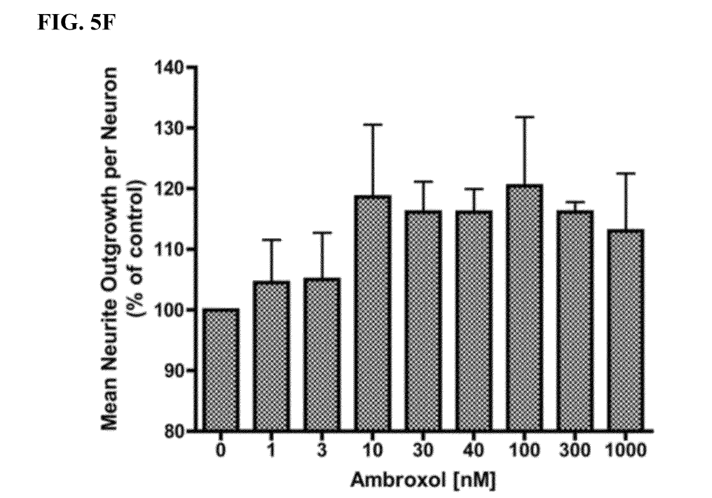

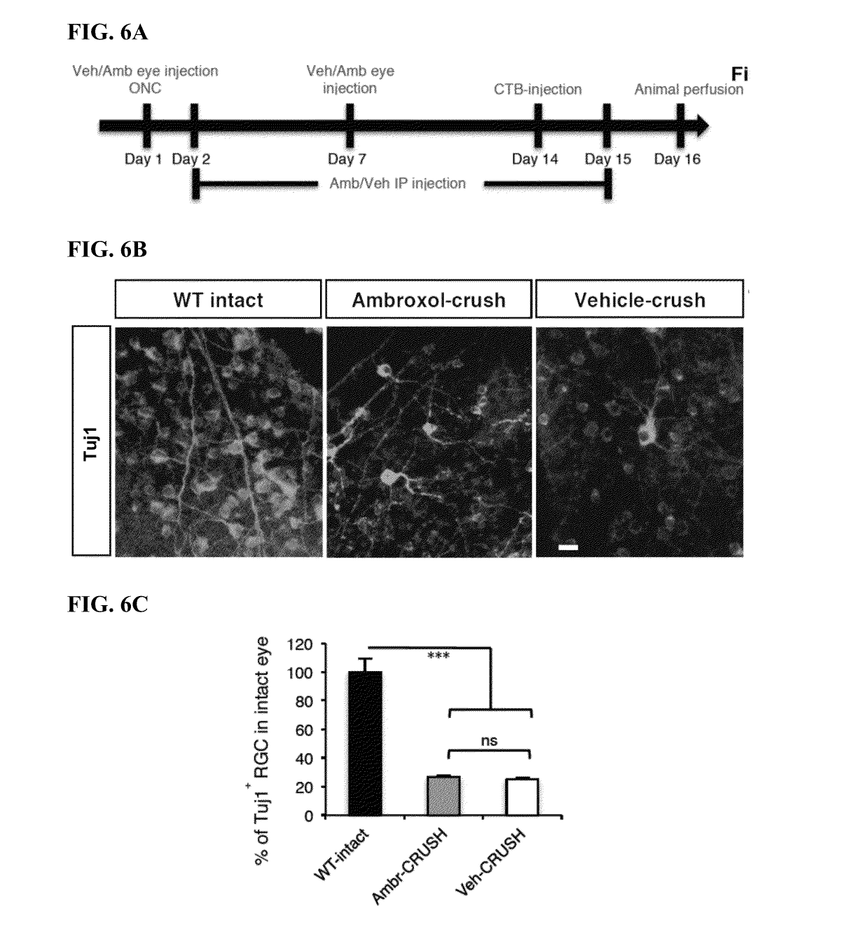

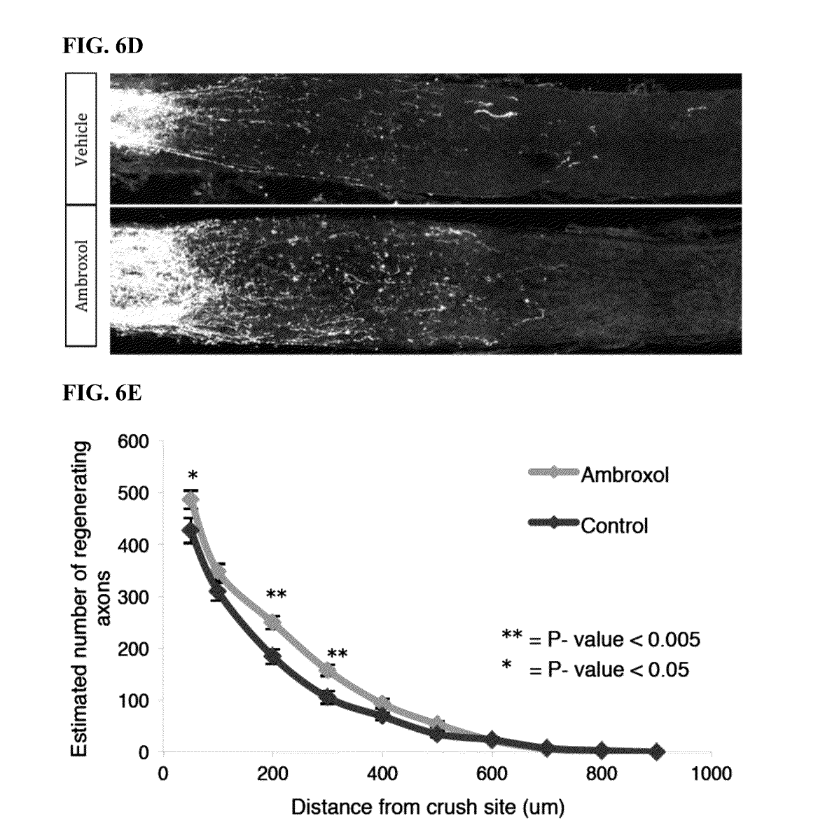

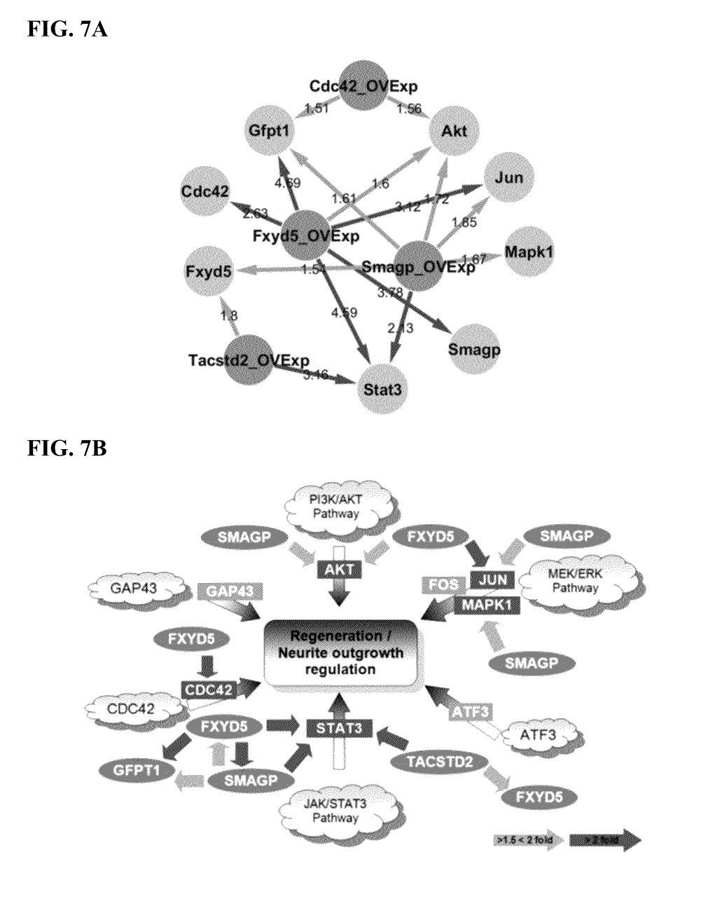

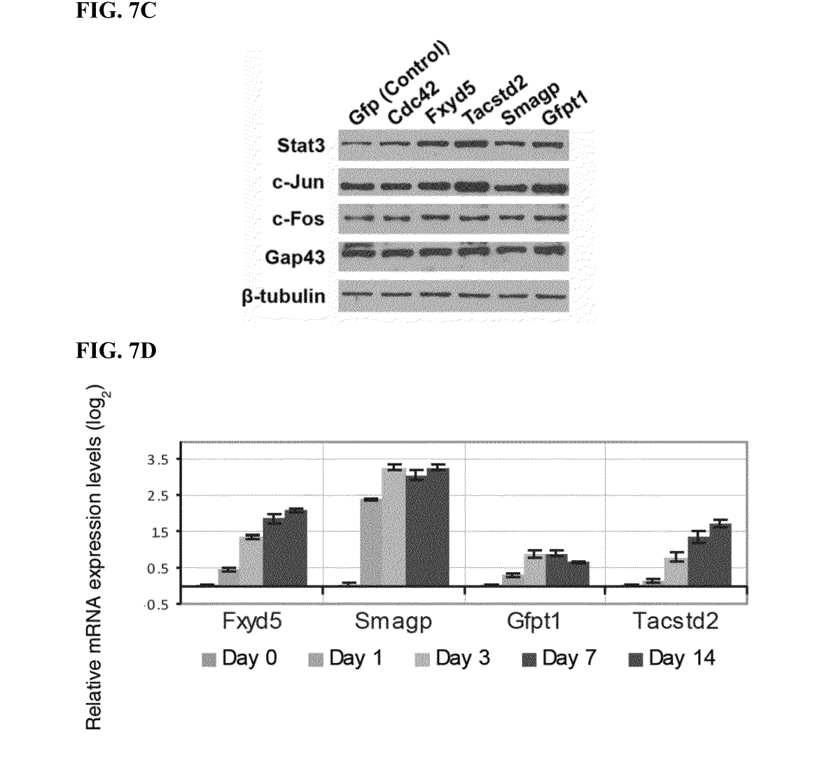

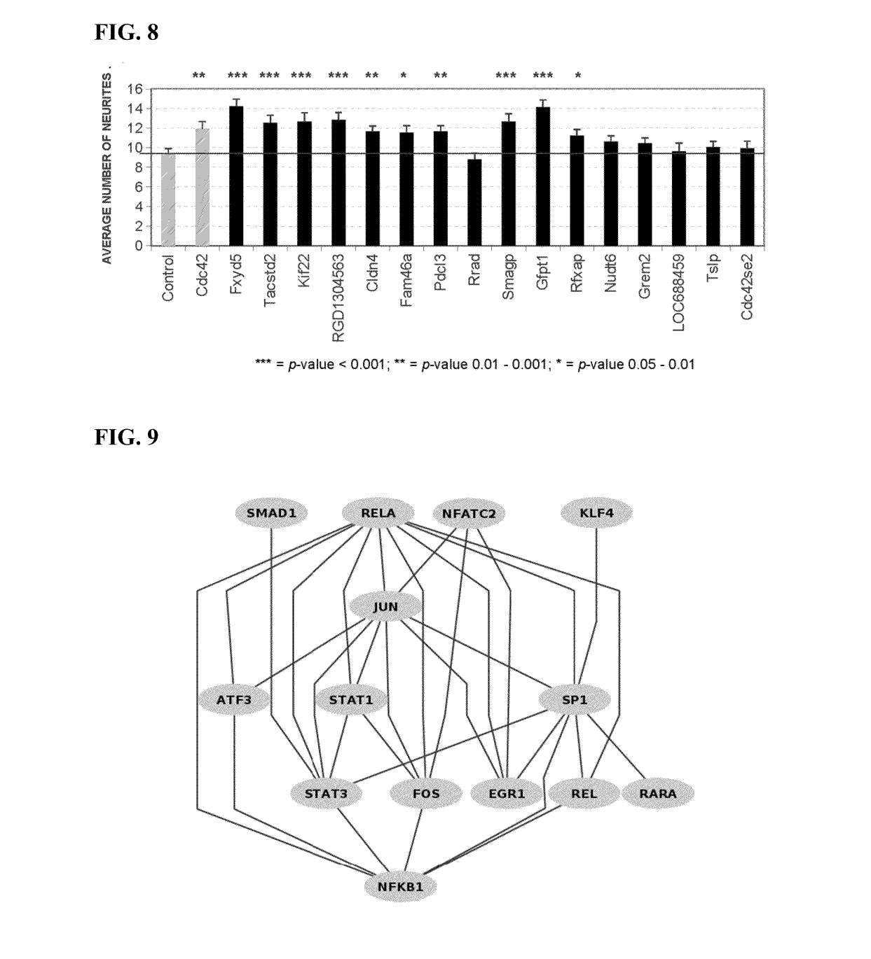

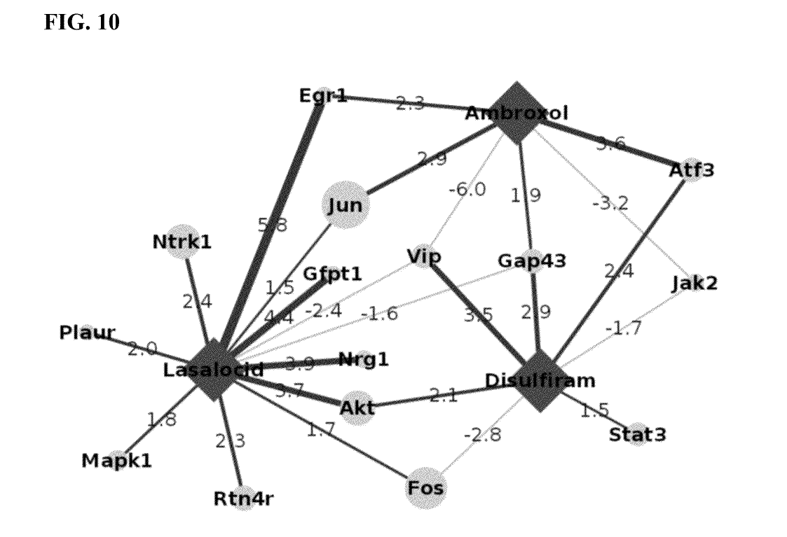

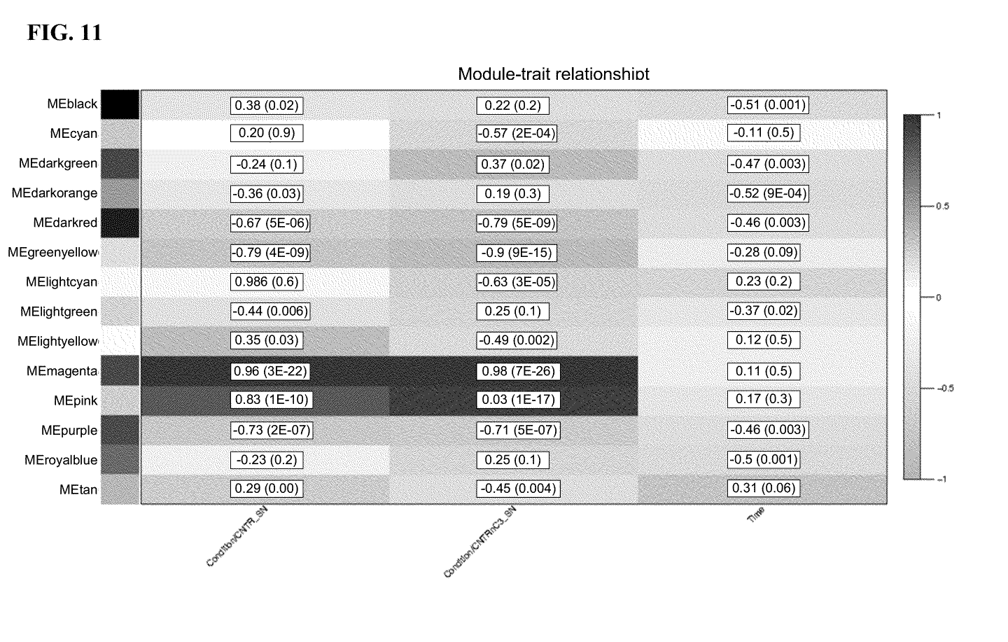

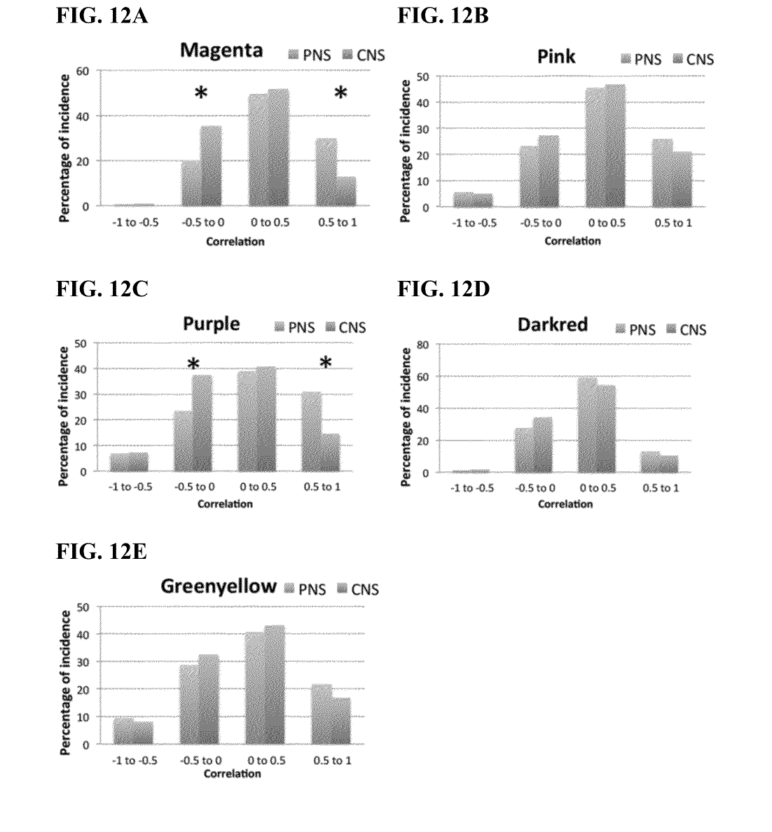

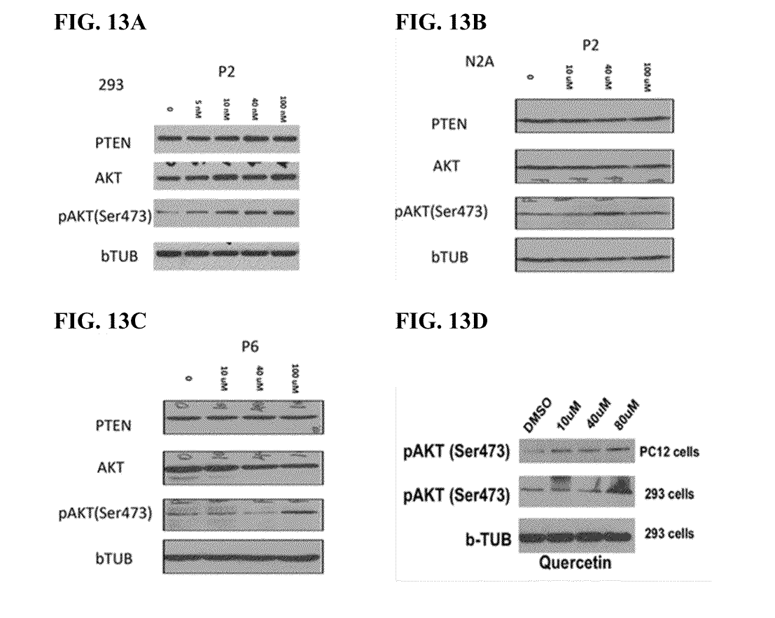

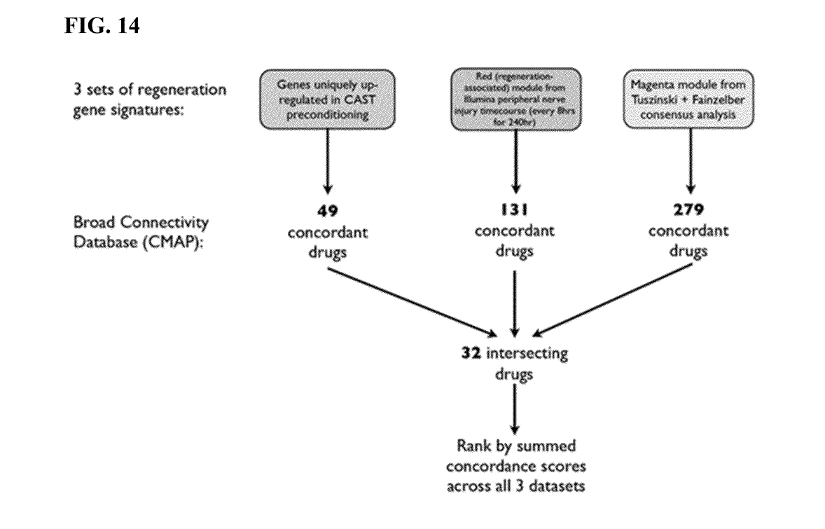

The gene expression levels of these 18 TFs (plus other TFs from literature) were examined in independent PNS and compared with CNS injury samples (spinal cord injury), where neuronal regeneration is limited relative to PNS injury. Regardless of the injury model studied, these TFs were co-expressed and significantly up-regulated after PNS injury in multiple data sets (FIG. 5A and FIG. 8). In four independent CNS injury datasets (spinal cord injury—mild, moderate, severe and complete transection) the levels of these TFs were significantly variable or down-regulated (FIG. 5A) (non-parametric Kruskai-Wallis, P<2.40E-05; FIG. 8). These independent experiments provide strong additional evidence that the coordinate regulation of these TF is related to nerve outgrowth after injury.

In many cases, TF regulation of gene expression is activated in a cooperative way mediated by direct physical contact between two or more TFs forming homodimers, heterodimers or larger transcriptional complexes (Ravasi et al. 2010). The over-represented TFs were tested to determine whether they physically interact with each other. Indeed, there is significant experimental support for this, for example, including interactions between ATF3, c-Jun, STAT3 and SP1 (Kiryu-Seo et al. 2008), suggesting that transcriptional regulation of the RAGs during the PNS injury is facilitated by the joint activity of these TFs as observed by analysis of their expression levels. Moreover, it has been shown that the transcription factor SP1, bound to the promoter region, recruits ATF3, c-Jun, and STAT3 and physically interacts with them to regulate gene-expression to obtain the requisite synergistic effect (Kiryu-Seo et al. 2008). Given the minimal regenerative effect after individual over-expression of these TFs in isolation (example: ATF3—(Seijffers et al. 2007); STAT3—(Bareyre et al. 2011)), these data suggest that combined synergistic effect of these over-represented, co-expressed and physically interacting TFs are likely necessary to create a neuronal growth state to effectively recover after injury. Hence targeting the neuronal growth state networks rather than a single gene would be a better approach for effective recovery after injury.

Regulation of Neurite Outgrowth in DRG Neurons Using Small Molecules.

The gene expression signatures were used to identify FDA approved drugs (Lamb 2007) that may modulate the signaling pathways and gene expression necessary for regeneration. The gene expression levels from the identified candidate up and down-regulated RAG co-expression networks and the PPI network were used as a signature with which a database of drug-related expression profiles known as the Connectivity Map (Lamb 2007; Lamb et al. 2006) was queried. Based on the results obtained from the pattern matching algorithm present within the Connectivity Map database, the top three matching expression patterns were chosen based on the connectivity and specificity score, which represented the drugs (ambroxol, lasalocid, and disulfiram). The identified compounds were tested for their effect on gene expression levels of regeneration-associated marker genes and to examine the effect on neurite outgrowth in DRG neurons. All the 3 drugs showed significant changes in the gene-expression levels of marker RAGs, but only ambroxol showed significant enhancement of axonal outgrowth in DRG neurons. We also observed that ambroxol produced significant dose response increase in a high-throughput neurite outgrowth assay using DRG neurons from Thy1-YFP reporter mice (FIG. 5D-F). Since the original pattern was derived from non-neuronal cell lines (30), we tested if ambroxol can change the gene-expression levels of these markers in DRG neurons. Ambroxol treated DRG neurons showed significant differential expression of 8 genes (P-value<0.05) among 14 randomly tested genes from the M77 (FIGS. 5A, 5C). Interestingly, this included 5 of the core hub transcription factors (ATF3, FOS, JUN, SMAD1 and SP1) in the M77 network (FIGS. 5A, 5C). Interestingly, GO enrichment analysis of down-regulated RAG co-expression modules revealed various categories related to ion/gated channels were significant enriched which included Na⁺ and Ca²⁺ channels. Of note, ambroxol is a potent blocker of neuronal Na⁺ and Ca²⁺ channels (Weiser 2008) and in animals models, ambroxol effectively suppresses symptoms of peripheral neuropathic pain (Gaida et al. 2005) and symptoms of neuropathic spinal cord injury pain in rats (Hama et al. 2010). But, it had no previous relationship to regeneration.

Table 5 following discloses analysis of transcription-factor binding-sites (TFBS) enrichment. For estimation of TFBSs enrichment in the identified corresponding module genelist (genes having ≧0.5 average connectivity) promoter sequences (1000 bp upstream from transcription start site), P-values were obtained relative to three background datasets: 1000-bp sequences upstream of all rat genes, rat CpG islands and rat chromosome 20 sequence. Last column represents PubMed association/co-occurrence of corresponding TF with the tags-neuronal regeneration, axonal regeneration, nerve injury.

TABLE 5
Analysis of TFBS.

| | | | | | |
|---|---|---|---|---|---|
| Enriched Transcription | Raw- | | | | |
| Factor | Score | P-value_DB | P-value_CpG | P-value_Chro20 | PubMed_Asso |

Magenta (195 genes, 195000 bp, 51.7% C + G)

| | | | | | |
|---|---|---|---|---|---|
| MA0079.2_SP1 | 306 | 0.000 | 0.000 | 0.000 | Asso |
| MA0039.2_Klf4 | 166 | 0.000 | 0.000 | 0.000 | Asso |
| MA0152.1_NFATC2 | 86.6 | 0.025 | 0.024 | 0.000 | Asso |
| MA0137.2_STAT1 | 39.4 | 0.001 | 0.000 | 0.000 | Asso |
| MA0144.1_Stat3 | 29.9 | 0.004 | 0.000 | 0.003 | Asso |
| MA0099.2_AP1 | 18.2 | 0.001 | 0.000 | 0.004 | Asso |
| MA0101.1_REL | 7.13 | 0.005 | 0.004 | 0.002 | Asso |
| MA0107.1_RELA | 6.74 | 0.004 | 0.007 | 0.001 | Asso |
| MA0162.1_Egr1 | 4.93 | 0.013 | 0.000 | 0.002 | Asso |
| MA0108.2_TBP | 2.81 | 0.031 | 0.976 | 0.002 | Asso |

Pink (30 genes, 30000 bp, 48.1% C + G)

| | | | | | |
|---|---|---|---|---|---|
| MA0152.1_NFATC2 | 17.4 | 0.008 | 0.028 | 0.000 | Asso |
| MA0157.1_FOXO3 | 5.98 | 0.047 | 0.021 | 0.024 | Asso |

Purple (127 genes, 127000 bp, 53.7% C + G)

| | | | | | |
|---|---|---|---|---|---|
| MA0079.2_SP1 | 192 | 0.001 | 0.000 | 0.000 | Asso |
| MA0039.2_Klf4 | 83.7 | 0.014 | 0.000 | 0.000 | Asso |
| MA0145.1_Tcfcp2l1 | 18 | 0.000 | 0.000 | 0.001 | Non-Asso |
| MA0138.2_REST | 14 | 0.003 | 0.011 | 0.000 | Asso |
| MA0141.1_Esrrb | 11.2 | 0.032 | 0.000 | 0.038 | Non-Asso |
| MA0014.1_Pax5 | 10.8 | 0.002 | 0.027 | 0.003 | Non-Asso |

Darkred (30 genes, 30000 bp, 48.8% C + G)

| | | | | | |
|---|---|---|---|---|---|
| MA0079.2_SP1 | 56.9 | 0.003 | 0.000 | 0.001 | Asso |
| MA0146.1_Zfx | 5.93 | 0.048 | 0.008 | 0.004 | Asso |
| MA0038.1_Gfi | 4.53 | 0.042 | 0.018 | 0.010 | Non-Asso |
| MA0101.1_REL | 2.2 | 0.050 | 0.039 | 0.020 | Asso |
| MA0107.1_RELA | 1.94 | 0.025 | 0.021 | 0.015 | Asso |

Greenyellow (32 genes, 32000 bp, 44.8% C + G)

| | | | | | |
|---|---|---|---|---|---|
| MA0079.2_SP1 | 48.7 | 0.043 | 0.000 | 0.000 | Asso |
| MA0149.1_EWSR1-FLI1 | 15.3 | 0.015 | 0.014 | 0.022 | Non-Asso |
| MA0088.1_znf143 | 14.8 | 0.007 | 0.000 | 0.000 | Non-Asso |
| MA0038.1_Gfi | 4.3 | 0.039 | 0.027 | 0.012 | Non-Asso |
| MA0162.1_Egr1 | 2.99 | 0.015 | 0.002 | 0.001 | Asso |

Table 6 following discloses analysis of transcription-factor binding-sites (TFBS) enrichment in rat, mouse and humans. TFBS enrichment analysis was performed for corresponding module-genelist promoter sequences obtained from rat, and their orthologous genes obtained from mouse and humans. TFs only enriched in any two organism were considered to be significantly enriched are mentioned in this table.

TABLE 6
Analysis of transcription-factor binding-sites
(TFBS) enrichment in rat, mouse and humans

| | | | | |
|---|---|---|---|---|
| | Raw- | | P- | |
| Enriched Transcription Factor | score | P-value_DB | value_CpG | P-value_Chro20 |

Up-regulated modules - RAT

| | | | | |
|---|---|---|---|---|
| MA0079.2_SP1 | 341 | 0.000 | 0.000 | 0.000 |
| MA0039.2_Klf4 | 174 | 0.000 | 0.000 | 0.000 |
| MA0152.1_NFATC2 | 105 | 0.004 | 0.009 | 0.000 |
| MA0137.2_STAT1 | 43 | 0.001 | 0.000 | 0.001 |
| MA0144.1_Stat3 | 28.7 | 0.017 | 0.000 | 0.012 |
| MA0099.2_AP1 | 16.9 | 0.001 | 0.000 | 0.005 |
| MA0107.1_RELA | 5.45 | 0.013 | 0.008 | 0.002 |
| MA0101.1_REL | 5.44 | 0.024 | 0.017 | 0.006 |
| MA0108.2_TBP | 3.29 | 0.035 | 0.992 | 0.001 |

Down-regulated modules - RAT

| | | | | |
|---|---|---|---|---|
| MA0079.2_SP1 | 299 | 0.000 | 0.000 | 0.000 |
| MA0057.1_MZF1_5-13 | 43.5 | 0.041 | 0.000 | 0.007 |
| MA0088.1_znf143 | 34.5 | 0.042 | 0.000 | 0.007 |
| MA0138.2_REST | 17.8 | 0.000 | 0.009 | 0.000 |
| MA0145.1_Tcfcp2l1 | 13.7 | 0.024 | 0.000 | 0.012 |
| MA0014.1_Pax5 | 12.8 | 0.002 | 0.034 | 0.005 |
| MA0162.1_Egr1 | 5.06 | 0.016 | 0.000 | 0.003 |
| MA0107.1_RELA | 4.97 | 0.013 | 0.008 | 0.002 |

Up-regulated modules - MOUSE

| | | | | |
|---|---|---|---|---|
| MA0079.2_SP1 | 401 | 0.000 | 0.000 | 0.000 |
| MA0039.2_Klf4 | 194 | 0.010 | 0.000 | 0.000 |
| MA0152.1_NFATC2 | 126 | 0.001 | 0.048 | 0.000 |
| MA0056.1_MZF1_1-4 | 91 | 0.024 | 0.000 | 0.010 |
| MA0137.2_STAT1 | 60.3 | 0.000 | 0.000 | 0.000 |
| MA0003.1_TFAP2A | 47 | 0.018 | 0.000 | 0.000 |
| MA0157.1_FOXO3 | 43.2 | 0.002 | 0.008 | 0.000 |
| MA0144.1_Stat3 | 41.9 | 0.000 | 0.000 | 0.001 |
| MA0099.2_AP1 | 17.2 | 0.004 | 0.000 | 0.036 |
| MA0107.1_RELA | 6.03 | 0.033 | 0.014 | 0.005 |

Down-regulated modules - MOUSE

| | | | | |
|---|---|---|---|---|
| MA0079.2_SP1 | 335 | 0.003 | 0.000 | 0.000 |
| MA0056.1_MZF1_1-4 | 77.2 | 0.036 | 0.000 | 0.030 |
| MA0065.2_PPARG::RXRA | 73.5 | 0.033 | 0.000 | 0.049 |
| MA0154.1_EBF1 | 51.1 | 0.003 | 0.000 | 0.019 |
| MA0138.2_REST | 28.5 | 0.000 | 0.000 | 0.000 |
| MA0162.1_Egr1 | 13.7 | 0.003 | 0.000 | 0.000 |
| MA0159.1_RXR::RAR_DR5 | 8.17 | 0.015 | 0.000 | 0.026 |

Up-regulated modules - HUMAN

| | | | | |
|---|---|---|---|---|
| MA0079.2_SP1 | 350 | 0.023 | 0.000 | 0.000 |
| MA0152.1_NFATC2 | 137 | 0.008 | 0.022 | 0.000 |
| MA0114.1_HNF4A | 42.4 | 0.031 | 0.003 | 0.018 |
| MA0109.1_Hltf | 41.7 | 0.001 | 0.010 | 0.000 |
| MA0148.1_FOXA1 | 39 | 0.018 | 0.020 | 0.000 |
| MA0040.1_Foxq1 | 28.9 | 0.015 | 0.032 | 0.000 |
| MA0144.1_Stat3 | 27.6 | 0.041 | 0.005 | 0.004 |
| MA0035.2_Gata1 | 8.52 | 0.044 | 0.016 | 0.002 |
| MA0159.1_RXR::RAR_DR5 | 7.17 | 0.012 | 0.003 | 0.014 |

Down-regulated modules - HUMAN

| | | | | |
|---|---|---|---|---|
| MA0079.2_SP1 | 278 | 0.042 | 0.000 | 0.000 |
| MA0056.1_MZF1_1-4 | 88.2 | 0.003 | 0.000 | 0.000 |
| MA0057.1_MZF1_5-13 | 73.3 | 0.001 | 0.000 | 0.000 |
| MA0114.1_HNF4A | 42.1 | 0.007 | 0.000 | 0.002 |
| MA0098.1_ETS1 | 38.1 | 0.050 | 0.973 | 0.000 |

Ambroxol Enhances Neuronal Regeneration In Vivo.

Ambroxol was tested for the ability to enhance CNS regeneration in vivo after injury. The optic nerve regeneration effect in mice after crush injury was evaluated after treating with and without ambroxol. At two weeks after injury, there was a significant increase in axon regeneration in the animals treated with ambroxol when compared with the control animals. Indeed, the animals treated with ambroxol also showed significantly increased RGC survival after injury. In summary, a FDA approved drug—ambroxol accelerates and improves neural repair in the CNS. This compound is only known clinically to treat sore throat and as a mucolytic for coughs.

### Example 2

### Identification of Activities of RAGs

The current models for genetic manipulation for robust regeneration of injured corticospinal tract (CST) axons are limited to gene deletion models (Sun et al., 2011). CNS. To address this, several small molecules currently used in the clinical trials for other purposes or indications have been identified to activate RAGs herein. Indeed, these compounds inhibit proteins and genes known to repress RAGs. Herein, two proteins, PTEN and SOCS2, when inhibited, were identified to activate the core regeneration associated network. Thus effectively targeting the signaling networks responsible for central and peripheral regeneration, identifies target compounds that enhance significant level of CNS regeneration in optic nerve crush mice model.

A series of compounds were investigated and four (4) compounds were discovered as either PTEN inhibitors or SOCS3 inhibitor. Luteolin, and quercetin were discovered as PTEN inhibitors. Genistein and phentolamine were discovered as SOCS3 inhibitors.

The inhibition of PTEN was tested following the phosphorylated form of AKT. Western blot analysis of cultured human (293), mouse (N2A) and rat (PC12) cells treated with Luteolin (P2) and Quercetin (P6) in the presence of increasing concentrations of Luteolin (P2) or Quercetin (P6) showed increased expression level of phosphorylated form of AKT denoting PTEN inhibition.

Likewise, the inhibition of SOCS3 was tested following the phosphorylated form of STAT3. Western blot analysis of cultured human (293), mouse (N2A) and rat (PC12) cells treated with Genistein (S5) and Phentolamine (S8) in the presence of increasing concentration of Genistein (S5) and Phentolamine (S8) showed increased expression level of phosphorylated form of STAT3—denoting SOCS3 inhibition.

Testing the compounds for their ability to enhance RAG activity was performed using a screen of three gene expression data sets related to neural repair as described herein. The screen uses multiple independent data sets and finds drugs that cluster into 4 specific classes, indicating structure activity relationships.

Identification of the transcript differences between growing versus non-growing PNS and CNS neurons provides an opportunity to understand the inability of the terminally differentiated neurons in the adult CNS to lose their capacity to grow as they acquire their specific functions. Here, an applied systems biology approach on nerve injury time-series gene expression data was used to identify the genes/pathways which are responsible for the neuronal injury recovery in PNS. Based on these nerve injury datasets a RAG co-expression network was generated and identified significant modules which might be responsible for successful regenerative process after peripheral nerve injury. By validating these modules in various other nerve injury datasets (totally 13 independent datasets) the genes present within these modules showed similar gene expression pattern—demonstrating the involvement of common transcriptional response/signaling pathways underling the process of effective neuronal regeneration process after PNS injury (Abe et al. 2008). These modules showed significant enrichment (Benjamini corrected P-value<0.05) for multiple signaling pathways known to be associated with neuronal regeneration process, consistent with previous findings (Abe et al. 2008). In these RAG co-expression modules several TFs were significantly over-represented for their corresponding TFBS. Indeed, many of these TFs were already been reported to be involved in the nerve injury recovery process (Sun et al. 2010), providing additional level of confirmation of the RAG co-expression module. These TFs were hubs in the RAG co-expression network and in the PPI network. In silica removal of these TFs in the PPI network drastically reduced the distribution of network path length. This observation clearly demonstrates that these over-represented TFs influences the cross-talk mechanism between the enriched signaling pathways in the PPI network. Thus, for effective regeneration to occur after PNS nerve injury, a coordinated co-expression pattern of these TFs along with other RAGs was critical.

One would expect opposite/variable response of these TFs in CNS injury where neuronal regeneration is very limited. The gene expression levels of these TFs were significantly variable/down-regulated when compared to the PNS injury where these TFs were co-expressed/up-regulated after injury. These over-represented TFs are known to physically interact with each other. Over-expression of these TFs in a coordinated fashion in CNS injury model improves the extent of regenerative capacity after injury, and potentially therapeutic intervention. For example, constitutive expression of single TF (ATF3) increases axonal growth in the PNS after injury, but it does not overcome myelin inhibition in culture or enhance neuronal regeneration in the CNS (Seijffers et al. 2007). These findings suggests that coordinated co-expression of these over-represented TFs along with RAGs enhance the neuronal regeneration process in PNS and CNS after injury (Kiryu-Seo et al. 2008).

TFBS enrichment analysis was applied in the RAG co-expression modules, identifying seven over-represented TFs in the up-regulated modules (candidate transcriptional activators). These were enriched in both rat and mouse promoter regions demonstrating the phylogenetic conservation of the TFBS. All the seven TFs were previously associated with nerve injury/neuronal regeneration process providing additional level of confirmation of our results (Sun et al. 2010). When examined for TFBS enrichment in the down-regulated modules, MZF1 (candidate transcriptional repressor) was significantly enriched in the promoter regions of rat, mouse and human (high phylogenetic conservation of TFBS). Over-expression of MZF1 in adult DRG neurons results in significant enhancement in NOG relative to the controls. The involvement of MZF1 in nerve injury/neurite out-growth regulation has not been reported previously. These observations suggest there are additional unidentified candidates regulators (TFs and miRNAs) which are involved in the regeneration recovery process.

Additional candidate RAGs were screened for by applying two different systems biology approach. As a result, novel candidate RAGs were identified and validated, showing their over-expression increased, and their knockdown decreased, axonal outgrowth respectively, in adult DRG neurons (Fxyd5, Gfpt1, Smagp and Tacstd2). Without being bound by any theory, the mechanism by which four of these novel candidate RAGs induced NOG regulation results from RAGs up-regulating several marker signaling pathway genes and proteins involved in the regeneration process. By an approach targeting the entire core regeneration-associated network using small molecules which can recapitulate the conserved signaling pathways represented by the RAG co-expression modules differentially expressed after nerve injury, an FDA approved drug ambroxol was identified which significantly enhances NOG in DRG neurons and promotes CNS regeneration in vivo.

10 novel candidate RAGs were identified that significantly enhance neurite out-growth when over-expressed in adult mouse DRG neurons. Knock-down of candidate RAGs significantly reduces NOG. In addition, these candidate RAGs enhance NOG in DRG neurons by up-regulating several signaling pathway genes and proteins known to play a vital role in neuronal regeneration process. Based on the TFBS enrichment analysis the binding sites for MZF1 were enriched in down-regulated genes after nerve injury and over-expressing MZF1 in DRG neurons significantly enhanced NOG. By applying systems biology approach RAG co-expression modules were constructed exhibiting the following properties, (1) RAG modules consist of highly co-expressed and differentially regulated genes after PNS injury, (2) significant number of genes present within the up-regulated module were highly enriched for literature association (88 genes), (3) several signaling pathways related to neuronal regeneration were over-represented in the up-regulated module, (4) several regeneration associated TFs were over-presented for their corresponding TFBS in the promoter regions of the genes present within the module, (5) these over-represented TFs were acting as hubs in the module and in the PPI network, suggesting cross-talk with enriched signaling pathways. This was demonstrated by the drastic reduction in the distribution of the network path length after in silica removal of these TFs. These TFs were significantly variable/down-regulated in the CNS injury where regeneration is limited.

In terms of CNS extrinsic factors controlling neuronal-regeneration, various CNS myelin inhibitory components: Nogo, MAG, and OMgp have been tested for their inhibitory properties in vitro and in vivo in SCI models (Schnell et al. 1990; McKerracher et al. 1994; U. Bartsch et al. 1995; Bregman et al. 1995; Ji et al. 2008). Individual knock-out of these genes has been implicated only in limited regeneration (Ferreira et al. 2012). Examination of triple knock-out mice for Nogo, MAG, and OMgp for potential synergistic inhibitory effect of these three proteins on axonal regeneration in injured adult CNS has produced contradicting results (Cafferty et al. 2010; J. K. Lee et al. 2010). It is known that limited regenerative capacity of the central branch can be rescued when the peripheral axon is damaged prior to, at the time of, or following the injury of the central one, and this is known as the conditioning effect (P M Richardson et al. 1984; Neumann et al. 1999). A conditioning lesion of peripheral branches, induced by harvesting a segment of sciatic nerve for transplantation into the spinal cord, increases the intrinsic growth state of central branches and promotes their lengthy regeneration in a peripheral nerve graft. This regeneration does not occur in the absence of a conditioning lesion, demonstrating the importance of intrinsic factors contributing to neuronal-regeneration. Hence, the limited regenerative potential of CNS neurons is due to the CNS microenvironment, which actively represses axonal regeneration after injury and due to a lack of intrinsic program potential for axonal regeneration, as opposed to what occurs in the PNS.

In past several decades of focus on intrinsic program has resulted in identification of several RAGs; including cytoskeletal proteins, cell adhesion and axon guidance molecules, transcription factors, trophic factors and their receptors (Rishal et al. 2010; Sun et al. 2010; Giger et al. 2010), and many of these genes are differentially expressed after nerve injury where its expression levels stays high up to 40 days post injury (Costigan et al. 2002). Earlier findings indicate that single RAG alone (e.g., ATF-3 or STAT3 or GAP-43) is not sufficient for effective successful regeneration (Seijffers et al. 2007; Bareyre et al. 2011; Bomze et al. 2001), demonstrating the necessary for combined and cooperative intrinsic regulation of many RAGs involved in various pathways for regeneration program to occur. An attempt to recapitulate this complex regeneration regulation to occur after PNS and CNS injury would be of great importance for successful regeneration. At the same time it would be a tedious process to recapitulate since complete regeneration associated signaling pathways are not yet completely understood.

The compound and methods herein, were applied using two different approaches to recapitulate this complex network in order to establish the active intrinsic growth state of axons. Herein small-molecules were identified which can target and recapitulate the regeneration-associated network, and by global regulators were identified which play a vital role in regulating the regeneration-associated network responsible for PNS regeneration. By applying these two approaches a set of FDA approved drugs that enhance neurite out-growth in DRG neurons in vitro and promoted CNS regeneration in vivo were identified. Also identified are a series of critical regulators to recapitulate the core gene-network for successful regeneration to occur. An approach for targeting the transcriptional regulators that regulate these complex and coordinate regeneration signaling pathways poses as one mechanism to achieve successful regeneration. The over-expression of these transcription regulators (TFs over-represented in the RAG co-expression networks) in coordinated fashion represents a promising approach to understand the regeneration mechanism after PNS and CNS injury.

Inter alia, we analyzed mRNA expression generated in multiple PNS injury models from dorsal root ganglion (DRG) neurons in adult rats. Gene expression alterations across different nerve injury models involving peripheral and central nervous systems at various time points were analyzed. A core regeneration associated gene network in the PNS was identified, and validated the growth promoting function of a subset of RAG candidates in adult DRG mouse neurons. Integration of protein—protein interactions with RNA co-expression network analysis identified a core regeneration associated gene co-expression module that was highly enriched for a core set of hub genes including known transcription factors (TFs) known to promote neurite outgrowth. MZF1 not previously associated with neurite outgrowth, enhanced neurite outgrowth in DRG mouse neurons was identified using this approach. Pathway analysis indicates that rather than acting in isolation, these enriched TFs provide cross-talk between the over-represented signaling pathways responsible for neuronal regeneration after PNS injury; It is notable that these TFs expression levels are coordinately up-regulated following PNS injury, but are not similarly coordinated after CNS injury, suggesting a core set of TFs whose coordinate regulation may be necessary for process outgrowth.

Integrative analyses of multiple datasets of gene expression changes occurring during peripheral regeneration after nerve injury was performed. From this a core set of gene expression modules associated with the presence of regeneration was identified. One of these modules (magenta), included 22% of the known regeneration-associated genes up-regulated after nerve injury, as well as many novel genes not annotated for regeneration. 10 of these novel genes were tested in vitro and demonstrate increased neurite formation, validating the magenta module as being enriched in regeneration-associated genes. Promoter sequences of genes in the magenta module were inspected, the core transcriptional program needed to induce nerve growth. This core network was extended by integrating experimentally validated protein-protein interaction data, The identified key transcription factors (TF's) act as master regulators of regeneration during the nerve injury process. We found that many TFs are expressed in a coordinated fashion in the peripheral nervous system (PNS) (where regeneration occurs) but not in central nervous system (CNS) (where regeneration fails) after injury. This core regeneration-associated network was used to identify target small molecules that promote regeneration using a pattern matching algorithm. By targeting the identified core regeneration-associated network after PNS injury, a series of compounds (e.g. ambroxol—an FDA approved drug) were found enhance neurite outgrowth in dorsal root ganglion neurons and CNS regeneration in vivo.

### Example 3

### Ambroxol Enhances CNS Regeneration In Vivo

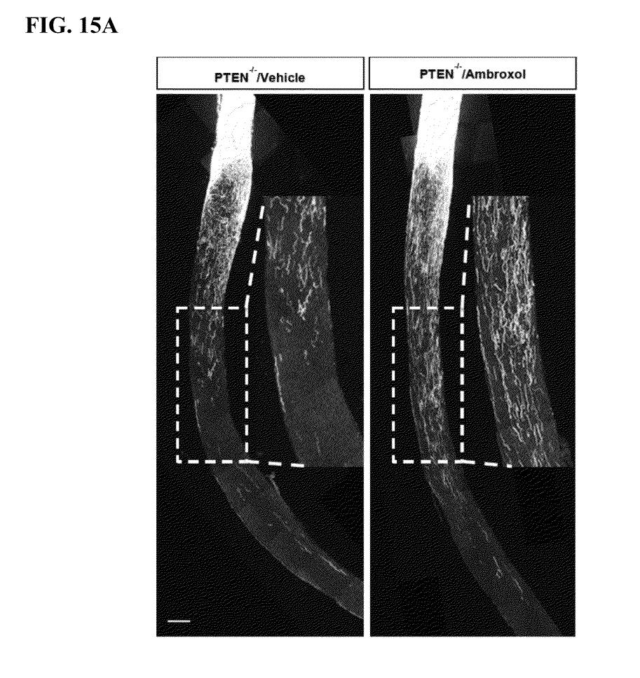

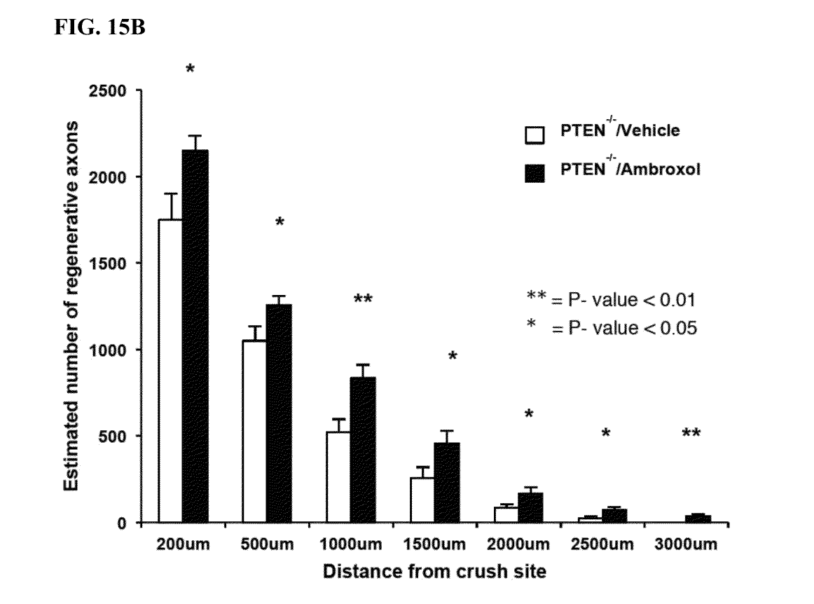

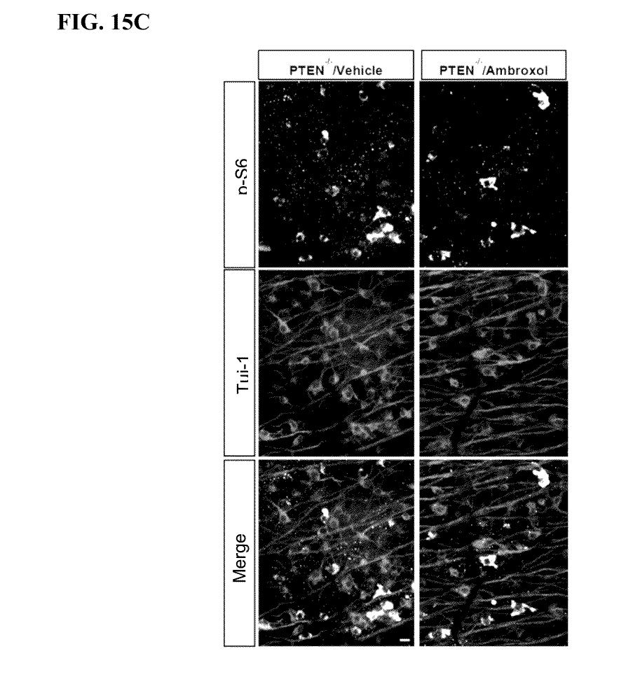

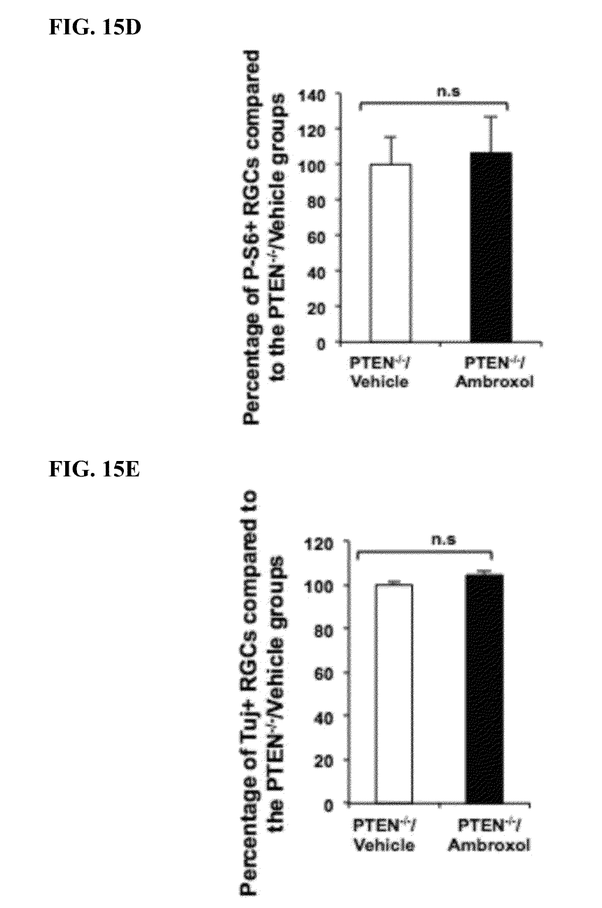

Another prediction from our network analyses is that appropriate co-regulation of the core regeneration associated module, M280, which does not normally occur in CNS injury, might augment CNS regeneration. Without wishing to be bound by any theory, since ambroxol recapitulates many of the core expression changes in the M280, we reasoned that it would promote CNS regeneration. Optic nerve (ON) regeneration has become a standard model for CNS regeneration [Sun et al., 2011], so we examined ON regeneration in C57BL/6 mice after a crush injury following treatment with ambroxol (Experimental Procedures). We observed limited but significant increase in axon regeneration beyond the site of the lesion (>1.5 fold increase between 200-500 um; p-value less than 0.04) after 2 weeks in animals treated with ambroxol compared with control animals, confirming the predictive properties of the approach. Next we hypothesized that, ambroxol combined with another treatment paradigm would further enhance axonal regeneration after injury. For that, we examined the combinatorial approach of ambroxol treatment in PTEN knockout mice after optic nerve crush, where PTEN^{-/-} mice have previously shown to improve axonal regeneration [Park et al., 2008]. We observed enhanced regeneration beyond the site of the lesion (>2.9 fold increase at 2500 um; p-value 0.02) after 2 weeks in PTEN^{-/-} animals treated with ambroxol compared with control animals (FIGS. 15A-15E), confirming that the combinatorial approach further enhances regeneration. These data confirm our hypothesis and provide strong independent evidence that the activation of the identified core network can enhance CNS nerve outgrowth after injury, providing an opportunity to explore the identified networks, transcriptions factors and small molecule as targets for further investigation to enhance CNS regeneration.

### REFERENCES

Abe, N., and Cavalli, V. (2008). Nerve injury signaling. Current opinion in neurobiology 18, 276-83.
Afshari, F. T., Kappagantula, S., and Fawcett, J. W. (2009). Extrinsic and intrinsic factors controlling axonal regeneration after spinal cord injury. Expert reviews in molecular medicine 11, e37.
Barabasi, A.-L., Gulbahce, N., and Loscalzo, J. (2011). Network medicine: a network-based approach to human disease. Nature Reviews Genetics 12, 56-68.
Barabasi, A.-L., and Oltvai, Z. N. (2004). Network biology: understanding the cell's functional organization. Nature reviews. Genetics 5, 101-13.
Bareyre, F. M., Garzorz, N., Lang, C., Misgeld, T., and BOning, H. (2011). In vivo imaging reveals a phase-specific role of STAT3 during central and peripheral nervous system axon regeneration. Direct, 6-11.
Bartsch, U. et al. (1995). Lack of evidence that myelin-associated glycoprotein is a major inhibitor of axonal regeneration in the CNS. Neuron 15, 1375-81.
Beggs, S., and A, M. W. S. (2006). Neuropathic Pain: Symptoms, Models, and Mechanisms. Pain 301, 289-301.
Bomze, H. M., Bulsara, K. R., Iskandar, B. J., Caroni, P., and Skene, J. H. (2001). Spinal axon regeneration evoked by replacing two growth cone proteins in adult neurons. Nature neuroscience 4, 38-43.
Bregman, B. S., Kunkei-Bagden, E., Schnell, L., Dai, H. N., Gao, D., and Schwab, M. E. (1995). Recovery from spinal cord injury mediated by antibodies to neurite growth inhibitors. Nature 378, 498-501.
Busch, S. A., and Silver, J. (2007). The role of extracellular matrix in CNS regeneration. Current opinion in neurobiology 17, 120-7.
Cafferty, W. B. J., Duffy, P., Huebner, E., and Strittmatter, S. M. (2010). MAG and OMgp synergize with Nogo-A to restrict axonal growth and neurological recovery after spinal cord trauma. The Journal of neuroscience: the official journal of the Society for Neuroscience 30, 6825-37.
Costigan, M. et al. (2002). Replicate high-density rat genome oligonucleotide microarrays reveal hundreds of regulated genes in the dorsal root ganglion after peripheral nerve injury. BMC neuroscience 3, 16.
Cubas, R., Zhang, S., Li, M., Chen, C., and Yao, Q. (2010). Trop2 expression contributes to tumor pathogenesis by activating the ERK MAPK pathway. Molecular cancer 9, 253.
Ferreira, L. M. R., Floriddia, E. M., Quadrato, G., and Giovanni, S. Di (2012). Neural Regeneration: Lessons from Regenerating and Non-regenerating Systems. Molecular neurobiology.
Filbin, M. T. (2003). Myelin-associated inhibitors of axonal regeneration in the adult mammalian CNS. Nature reviews. Neuroscience 4, 703-13.
Gaida, W., Klinder, K., Arndt, K., and Weiser, T. (2005). Ambroxol, a Nav1.8-preferring Na(+) channel blocker, effectively suppresses pain symptoms in animal models of chronic, neuropathic and inflammatory pain. Neuropharmacology 49, 1220-7.
Geschwind, D. H., and Konopka, G. (2009). Neuroscience in the era of functional genomics and systems biology. Nature 461, 908-15.
Giger, R. J., Hollis, E. R., and Tuszynski, M. H. (2010). Guidance molecules in axon regeneration. Cold Spring Harbor perspectives in biology 2, a001867.
Hama, A. T., Plum, A. W., and Sagen, J. (2010). Antinociceptive effect of ambroxol in rats with neuropathic spinal cord injury pain. Pharmacology, biochemistry, and behavior 97, 249-55.
Herdegen, T., Bastmeyer, M., Bahr, M., Stuermer, C., Bravo, R., and Zimmermann, M. (1993). Expression of JUN, KROX, and CREB transcription factors in goldfish and rat retinal ganglion cells following optic nerve lesion is related to axonal sprouting. Journal of neurobiology 24, 528-43.
Ishikawa, K., Tanaka, M., Black, J. A., and Waxman, S. G. (1999). Changes in expression of voltage-gated potassium channels in dorsal root ganglion neurons following axotomy. Muscle & nerve 22, 502-7.
Ji, B. et al. (2008). Assessment of functional recovery and axonal sprouting in oligodendrocyte-myelin glycoprotein (OMgp) null mice after spinal cord injury. Molecular and cellular neurosciences 39, 258-67.
Kiryu-Seo, S., Kato, R., Ogawa, T., Nakagomi, S., Nagata, K., and Kiyama, H. (2008). Neuronal injury-inducible gene is synergistically regulated by ATF3, c-Jun, and STAT3 through the interaction with Sp1 in damaged neurons. The Journal of biological chemistry 283, 6988-96.
Konopka, G. et al. (2009). Human-specific transcriptional regulation of CNS development genes by FOXP2. Nature 462, 213-7.
Lamb, J. (2007). The Connectivity Map: a new tool for biomedical research. Nature reviews. Cancer 7, 54-60.
Lamb, J. et al. (2006). The Connectivity Map: using gene-expression signatures to connect small molecules, genes, and disease. Science (New York, N.Y.) 313, 1929-35.
Langfelder, P., and Horvath, S. (2007). Eigengene networks for studying the relationships between co-expression modules. BMC systems biology 1, 54.
Langfelder, P., and Horvath, S. (2008). WGCNA: an R package for weighted correlation network analysis. BMC bioinformatics 9, 559.
Lee, J. K., Geoffroy, C. G., Chan, A. F., Tolentino, K. E., Crawford, M. J., Leal, M. A., Kang, B., and Zheng, B. (2010). Assessing spinal axon regeneration and sprouting in Nogo-, MAG-, and OMgp-deficient mice. Neuron 66, 663-70.
Liu, K. et al. (2010). PTEN deletion enhances the regenerative ability of adult corticospinal neurons. Nature neuroscience 13, 1075-81.
McKerracher, L., David, S., Jackson, D. L., Kottis, V., Dunn, R. J., and Braun, P. E. (1994). Identification of myelin-associated glycoprotein as a major myelin-derived inhibitor of neurite growth. Neuron 13, 805-11.
Michaelevski, I. et al. (2010). Signaling to transcription networks in the neuronal retrograde injury response. Science signaling 3, ra53.
Moore, D. L., Blackmore, M. G., Hu, Ying, Kaestner, K. H., Bixby, J. L., Lemmon, V. P., and Goldberg, J. L. (2009). KLF family members regulate intrinsic axon regeneration ability. Science (New York, N.Y.) 326, 298-301.
Nam, J.-S., Hirohashi, S., and Wakefield, L. M. (2007). Dysadherin: a new player in cancer progression. Cancer letters 255, 161-9.
Neumann, S., and Woolf, C J (1999). Regeneration of dorsal column fibers into and beyond the lesion site following adult spinal cord injury. Neuron 23, 83-91.
Okita, K., Ichisaka, T., and Yamanaka, S. (2007). Generation of germline-competent induced pluripotent stem cells. Nature 448, 313-7.
Oldham, M. C., Konopka, G., Iwamoto, K., Langfelder, P., Kato, T., Horvath, S., and Geschwind, D. H. (2008). Functional organization of the transcriptome in human brain. Nature neuroscience 11, 1271-82.
Park, I.-H., Zhao, R., West, J. A., Yabuuchi, A., Huo, H., Ince, T. A., Lerou, P. H., Lensch, M. W., and Daley, G. Q. (2008). Reprogramming of human somatic cells to pluripotency with defined factors. Nature 451, 141-6.
Park, Kevin Kyungsuk et al. (2008). Promoting axon regeneration in the adult CNS by modulation of the PTEN/mTOR pathway. Science (New York, N.Y.) 322, 963-6.
Portales-Casamar, E. et al. (2010). JASPAR 2010: the greatly expanded open-access database of transcription factor binding profiles. Nucleic acids research 38, D105-10.
Qiu, J., Cafferty, W. B. J., McMahon, S. B., and Thompson, S. W. N. (2005). Conditioning injury-induced spinal axon regeneration requires signal transducer and activator of transcription 3 activation. The Journal of neuroscience: the official journal of the Society for Neuroscience 25, 1645-53.
Raivich, G. et al. (2004). The AP-1 transcription factor c-Jun is required for efficient axonal regeneration. Neuron 43, 57-67.
Ravasi, T. et al. (2010). An atlas of combinatorial transcriptional regulation in mouse and man. Cell 140, 744-52.
Richardson, P M, and lssa, V. M. (1984). Peripheral injury enhances central regeneration of primary sensory neurones. Nature 309, 791-3.
Rishal, I., and Fainzilber, M. (2010). Retrograde signaling in axonal regeneration. Experimental neurology 223, 5-10.
Schnell, L., and Schwab, M. E. (1990). Axonal regeneration in the rat spinal cord produced by an antibody against myelin-associated neurite growth inhibitors. Nature 343, 269-72.
Seijffers, R., Mills, C. D., and Woolf, Clifford J (2007). ATF3 increases the intrinsic growth state of DRG neurons to enhance peripheral nerve regeneration. The Journal of neuroscience: the official journal of the Society for Neuroscience 27, 7911-20.
Shim, S., and Ming, G. (2010). Roles of channels and receptors in the growth cone during PNS axonal regeneration. Experimental neurology 223, 38-44.
Silver, J., and Miller, J. H. (2004). Regeneration beyond the glial scar. Nature reviews. Neuroscience 5, 146-56.
Smith, G. M., and Onifer, S. M. (2010). Construction of pathways to promote axon growth within the adult central nervous system. Brain research bulletin.
Smith, P. D., Sun, F., Park, Kevin Kyungsuk, Cai, B., Wang, C., Kuwako, K., Martinez-Carrasco, I., Connolly, L., and He, Z. (2009). SOCS3 deletion promotes optic nerve regeneration in vivo. Neuron 64, 617-23.
Son, E. Y., Ichida, J. K., Wainger, B. J., Toma, J. S., Rafuse, V. F., Woolf, Clifford J, and Eggan, K. (2011). Conversion of mouse and human fibroblasts into functional spinal motor neurons. Cell stem cell 9, 205-18.
Sun, F. et al. (2011). Sustained axon regeneration induced by co-deletion of PTEN and SOCS3. Nature 480, 372-5.
Sun, F., and He, Z. Neuronal intrinsic barriers for axon regeneration in the adult CNS Fang Sun and Zhigang He. Current Opinion in Neurobiology 20, 510-518.
Sun, F., and He, Z. (2010). Neuronal intrinsic barriers for axon regeneration in the adult CNS. Current opinion in neurobiology 20, 510-8.
Takahashi, K., and Yamanaka, S. (2006). Induction of pluripotent stem cells from mouse embryonic and adult fibroblast cultures by defined factors. Cell 126, 663-76.
Tarbé, N. G., Rio, M.-C., and Weidle, U. H. (2004). SMAGP, a new small trans-membrane glycoprotein altered in cancer. Oncogene 23, 3395-403.
Weiser, T. (2008). Ambroxol: a CNS drug? CNS neuroscience & therapeutics 14, 17-24.
Wexler, E. M., Rosen, E., Lu, D., Osborn, G. E., Martin, E., Raybould, H., and Geschwind, D. H. (2011). Genome-wide analysis of a Wnt1-regulated transcriptional network implicates neurodegenerative pathways. Science signaling 4, ra65.
Wu, D., Yang, P., Zhang, X., Luo, J., Haque, M. E., Yeh, J., Richardson, Peter M, Zhang, Y., and Bo, X. (2009). Targeting a dominant negative rho kinase to neurons promotes axonal outgrowth and partial functional recovery after rat rubrospinal tract lesion. Molecular therapy: the journal of the American Society of Gene Therapy 17, 2020-30.
Yang, E.-K., Takimoto, K., Hayashi, Y., Groat, W. C. de, and Yoshimura, N. (2004). Altered expression of potassium channel subunit mRNA and alpha-dendrotoxin sensitivity of potassium currents in rat dorsal root ganglion neurons after axotomy. Neuroscience 123, 867-74.
Yiu, G., and He, Z. (2006). Glial inhibition of CNS axon regeneration. Nature reviews. Neuroscience 7, 617-27.
Yoon, C., Korade, Z., and Carter, B. D. (2008). Protein kinase A-induced phosphorylation of the p65 subunit of nuclear factor-kappaB promotes Schwann cell differentiation into a myelinating phenotype. The Journal of neuroscience: the official journal of the Society for Neuroscience 28, 3738-46.
Yu, J. et al. (2007). Induced pluripotent stem cell lines derived from human somatic cells. Science (New York, N.Y.) 318, 1917-20.

### SUPPLEMENTARY REFERENCES

[1]. W. K. Lim, K. Wang, C. Lefebvre, A. Califano, Comparative analysis of microarray normalization procedures: effects on reverse engineering gene networks. Bioinformatics 23, i282 (Jul. 1, 2007). [2]. B. Zhang, S. Horvath, A general framework for weighted gene coexpression network analysis. Statistical applications in genetics and molecular biology 4, Article17 (2005). [3]. M. C. Oldham, S. Horvath, D. H. Geschwind, Conservation and evolution of gene coexpression networks in human and chimpanzee brains. Proceedings of the National Academy of Sciences of the United States of America 103, 17973 (Nov. 21, 2006). [4]. P. Langfelder, S. Horvath, Eigengene networks for studying the relationships between co-expression modules. BMC systems biology 1, 54 (2007). [5]. W. Huang da, B. T. Sherman, R. A. Lempicki, Systematic and integrative analysis of large gene lists using DAVID bioinformatics resources. Nature protocols 4, 44 (2009). [6]. K. G. Becker et al., PubMatrix: a tool for multiplex literature mining. BMC bioinformatics 4, 61 (Dec. 10, 2003). [7]. S. V. Dongen, Graph Clustering Via a Discrete Uncoupling Process. SIAM J. Matrix Anal. Appl. 30, 121 (2008). [8]. J. Ernst, Z. Bar-Joseph, STEM: a tool for the analysis of short time series gene expression data. BMC bioinformatics 7, 191 (2006). [9]. E. Portales-Casamar et al., JASPAR 2010: the greatly expanded openaccess database of transcription factor binding profiles. Nucleic acids research 38, D105 (January, 2010). [10]. T. L. Bailey, C. Elkan, Fitting a mixture model by expectation maximization to discover motifs in biopolymers. Proceedings/ . . . International Conference on Intelligent Systems for Molecular Biology; ISMB. International Conference on Intelligent Systems for Molecular Biology 2, 28 (1994). [11]. M. Sena-Esteves, J. C. Tebbets, S. Steffens, T. Crombleholme, A. W. Flake, Optimized large-scale production of high titer lentivirus vector pseudotypes. Journal of virological methods 122, 131 (Dec. 15, 2004). [12]. M. C. Frith et al., Detection of functional DNA motifs via statistical overrepresentation. Nucleic acids research 32, 1372 (2004). [13]. J. Ye et al., Primer-BLAST: a tool to design target-specific primers for polymerase chain reaction. BMC bioinformatics 13, 134 (2012). [14]. A. Franceschini et al., STRING v9.1: protein-protein interaction networks, with increased coverage and integration. Nucleic acids research 41, D808 (January, 2013). [15]. J. Lamb et al., The Connectivity Map: using gene-expression signatures to connect small molecules, genes, and disease. Science 313, 1929 (Sep. 29, 2006). [16]. K. K. Park et al., Promoting axon regeneration in the adult CNS by modulation of the PTEN/mTOR pathway. Science 322, 963 (Nov. 7, 2008). [17]. A. Blesch et al., Conditioning lesions before or after spinal cord injury recruit broad genetic mechanisms that sustain axonal regeneration: superiority to camp-mediated effects. Experimental neurology 235, 162 (May, 2012). [18]. K. Kadoya et al., Combined intrinsic and extrinsic neuronal mechanisms facilitate bridging axonal regeneration one year after spinal cord injury. Neuron 64, 165 (Oct. 29, 2009). [19]. I. Michaelevski et al., Signaling to transcription networks in the neuronal retrograde injury response. Science signaling 3, ra53 (2010). [20]. M. Costigan et al., Replicate high-density rat genome oligonucleotide microarrays reveal hundreds of regulated genes in the dorsal root ganglion after peripheral nerve injury. BMC neuroscience 3, 16 (Oct. 25, 2002). [21]. M. Costigan et al., Multiple chronic pain states are associated with a common amino acid-changing allele in KCNS1. Brain: a journal of neurology 133, 2519 (September, 2010). [22]. C. H. Ma et al., The BMP coreceptor RGMb promotes while the endogenous BMP antagonist noggin reduces neurite outgrowth and peripheral nerve regeneration by modulating BMP signaling. The Journal of neuroscience: the official journal of the Society for Neuroscience 31, 18391 (Dec. 14, 2011). [23]. Y. Wang et al., Gene network revealed involvements of Birc2, Birc3 and Tnfrsf1a in anti-apoptosis of injured peripheral nerves. PloS one 7, e43436 (2012). [24]. G. Geeven et al., LLM3D: a log-linear modeling-based method to predict functional gene regulatory interactions from genome-wide expression data. Nucleic acids research 39, 5313 (July, 2011). [25]. D. Fischer, V. Petkova, S. Thanos, L. I. Benowitz, Switching mature retinal ganglion cells to a robust growth state in vivo: gene expression and synergy with RhoA inactivation. The Journal of neuroscience: the official journal of the Society for Neuroscience 24, 8726 (Oct. 6, 2004). [26]. S. Di Giovanni et al., Gene profiling in spinal cord injury shows role of cell cycle in neuronal death. Annals of neurology 53, 454 (April, 2003). [27]. J. Ryge et al., Transcriptional regulation of gene expression clusters in motor neurons following spinal cord injury. BMC genomics 11, 365 (2010).

Sequences

Primer pairs useful in the procedures disclosed herein include the following.

| GENE | PRIMER | | SEQ ID |
|---|---|---|---|
| NAME | NAME | SEQUENCE | NO: |
| AKT1 | AKT1_L1 | CCACGCTACTTCCTCCTCAA | 1 |
| | AKT1_R1 | CAGCGGATGATGAAGGTGTT | 2 |
| ATF3 | ATF3_MR_L | CCAGGTCTCTGCCTCAGAAG | 3 |
| | ATF3_MR_R | CATCTCCAGGGGTCTGTTGT | 4 |
| CASP3 | CASP3_RM_F | AAGATCACAGCAAAAGGAGCA | 5 |
| | CASP3_RM_R | GAGTTTCGGCTTTCCAGTCA | 6 |
| CDC42 | CDC42_L1 | TTGATACTGCAGGGCAAGAG | 7 |
| | CDC42_R1 | TGAGTTATCTCAGGCACCCA | 8 |
| EGR1 | EGR1_RM_F | ATTTTTCCTGAGCCCCAAAG | 9 |
| | EGR1_RM_R | CTGGGTACGGTTCTCCAGAC | 10 |
| FOS | FOS_MR_L | CCATGATGTTCTCGGGTTTC | 11 |
| | FOS_MR_R | TGTCACCGTGGGGATAAAGT | 12 |
| FXYD5 | FXYD5_2_L1 | TCCCAGATCCAGATCAAACC | 13 |
| | FXYD5_2_R1 | GGGTACCTTTCTTGCTGCTT | 14 |
| GABBR2 | GABBR2_RM_F | TGTTTGGCAGCAAGTACCAG | 15 |
| | GABBR2_RM_R | GCTGTGGAGTCTTCCCTGAG | 16 |
| GAP43 | GAP43_2_L1 | CAGGAAAGATCCCAAGTCCA | 17 |
| | GAP43_2_R1 | GAACGGAACATTGCACACAC | 18 |
| GFPT1 | GFPT1_L1 | TTGTGTCCCTCGTGATGTTT | 19 |
| | GFPT1_R1 | CCTTAATCAAGTCCGGCAGT | 20 |
| HTR3A | HTR3A_RM_F | CATGTATGCCATCCTCAACG | 21 |
| | HTR3A_RM_R | CCACGTCCACAAACTCATTG | 22 |
| JAK2 | JAK2_RM_F | GAAGCAGCAAGCATGATGAG | 23 |
| | JAK2_RM_R | GGCCACTCCAAGTTTCCATA | 24 |
| JUN | JUN_L1 | ACATGCTCAGGGAACAGGTG | 25 |
| | JUN_R1 | TCAAAACGTTTGCAACTGCTG | 26 |
| KLF4 | KLF4_RM_F | GTGCCCCAAGATTAAGCAAG | 27 |
| | KLF4_RM_R | GTGACAGTCCCTGCTGTTCA | 28 |
| MAPK1 | MAPK1_L1 | TGCGCTTCAGACATGAGAAC | 29 |
| | MAPK1_R1 | TCTGTCTCCATGAGGTCCTGT | 30 |
| MYC | MYC_RM_F | GAGGGCCAAGTTGGACAGT | 31 |
| | MYC_RM_R | GCCTTTTCGTTGTTTTCCAA | 32 |
| MZF1 | MZF1_1M_F | CTCTGGTGAAGCTGGAGGAC | 33 |
| | MZF1_1M_R | GCACCTGTTCCTTGGAATGT | 34 |
| MZF1 | MZF1_2M_F | AAGATCCACACAGGCGAGAG | 35 |
| | MZF1_2M_R | AAGCTCTGGCCACACTCTGT | 36 |
| MZF1 | MZF1_3M_F | TCTAATCTCACCCAGCACCA | 37 |
| | MZF1_3M_R | TAGGGCTTCTCGCCAGTATG | 38 |
| NRG1 | NRG1_RM_F | CAAAAGAACCAAGCCCAATG | 39 |
| | NRG1_RM_R | TGCTGGGTTAGTCCTGCTCT | 40 |
| NTRK1 | NTRK1_RM_F | GTCTGGTGGGTCAGGGACTA | 41 |
| | NTRK1_RM_R | GGTGAAGATCTCCCAGAGCA | 42 |
| PLAUR | PLAUR_RM_F | CACAGCAGGTTTCCATAGCA | 43 |
| | PLAUR_RM_R | CCCAGCACATCTAAGCCTGT | 44 |
| RELA | RELA_RM_F | ATTAGCCAGCGAATCCAGAC | 45 |
| | RELA_RM_R | ATCTTGAGCTCGGCAGTGTT | 46 |
| RGS3 | RGS3_RM_F | CTGTTTGCCTACTCGGACCT | 47 |
| | RGS3_RM_R | CTTCTTCTGCTCCTGCGAGT | 48 |
| SCN11A | SCN11A_RM_F | ATTCTGGGGCCTTTTAATCC | 49 |
| | SCN11A_RM_R | CAATGAAGCCTCTTGCCAAT | 50 |
| SLC1A3 | SLC1A3_RM_F | GCCATTTTCATCGCTCAAGT | 51 |
| | SLC1A3_RM_R | CAGAAACCAGTCCACTGCAA | 52 |
| SMAD1 | SMAD1_RM_F | AGGCACAGCGAGTACAATCC | 53 |
| | SMAD1_RM_R | GAGTGAGGGTAGGTGCTGCT | 54 |
| SMAGP | SMAGP_Ll | GCGCTCATTGCAGTTGTTAT | 55 |
| | SMAGP_R1 | TCTGCAGGCTCATAGGTGAC | 56 |
| SP1 | SP1_RM_F | AAGCCCAGACAATCACCTTG | 57 |
| | SP1_RM_R | GCACCTGGATCCCTGAAGTA | 58 |
| STAT1 | STAT1_RM_F | CCCCATGGAAATCAGACAGT | 59 |
| | STAT1_RM_R | TCCTGGAGATTACGCTTGCT | 60 |
| STAT3 | STAT3_L1 | GGAGCAGAGATGTGGGAATG | 61 |
| | STAT3_R1 | TGGCAAGGAGTGGGTCTCTA | 62 |
| TACSTD2 | TACSTD2_ | CACCGCTGCTACTGCTACTG | 63 |
| | 2_L1 | | |
| | TACSTD2_ | GCAGGCACTTGGAAGTTAGC | 64 |
| | 2_R1 | | |
| TNK2 | TNK2_RM_F | GCCTGAAGACACGGACTTTC | 65 |
| | TNK2_RM_R | CAGCACTGGACCATGACATT | 66 |
| TRPV1 | TRPV1_RM_F | GCTAACGGGGACTTCTTCAA | 67 |
| | TRPV1_RM_R | TGTGTTATCTGCCACCTCCA | 68 |

1. A method of increasing neuronal growth in a subject in need thereof, said method comprising administering to said subject an effective amount of a compound capable of increasing expression of a regeneration-associated marker gene (RAG).

2. The method of claim 1, wherein said RAG is Fxyd5, Gfpt1, Smagp, Tacstd2, Kif22, RGD1304563, Cldn4, Fam46a, Rfxap or Pdcl3.

3. The method of claim 2, wherein said compound is capable of increasing expression of one, two, three, four, five, six, seven, eight, nine or all of Fxyd5, Gfpt1, Smagp, Tacstd2, Kif22, RGD1304563, Cldn4, Fam46a, Rfxap and Pdcl3.

4. The method of claim 1, wherein said RAG is Fxys5, Gfpt1, Smagp or Tacstd2.

5. The method of claim 4, wherein said compound is capable of increasing expression of one, two, three or all four of Fxys5, Gfpt1, Smagp and Tacstd2.

6. The method of claim 1, wherein said compound is capable of increasing the activity of a transcription factor selected from the group consisting of ATF3, CREB1, CTCF, EGR1, FOS, FOXI1, JUN, KLF4, MZF1, NFATC2, NFIL3, NFKB1, RARA, REL, RELA, REST, RORA, SMAD1, SOX11, SP1, STAT1, STAT3, and TFAP2A.

7. The method of claim 6, wherein said compound is capable of increasing the activity one, two, three, four, five, six, seven, eight, nine or all of ATF3, CREB1, CTCF, EGR1, FOS, FOXI1, JUN, KLF4, MZF1, NFATC2, NFIL3, NFKB1, RARA, REL, RELA, REST, RORA, SMAD1, SOX11, SP1, STAT1, STAT3, and TFAP2A.

8. The method of claim 1, wherein said compound is a Na⁺ channel blocker or a Ca²⁺ channel blocker.

9. The method of claim 1, wherein said compound suppresses symptoms of neuropathic pain.

10. The method of claim 1, wherein said compound suppresses symptoms of neuropathic spinal cord injury.

11. The method of claim 1, wherein said compound is ambroxol, an ambroxol derivative, or pharmaceutically acceptable salt thereof.

12. The method of claim 1, wherein said compound inhibits a RAG-repressor, thereby increasing expression of said RAG.

13. The method of claim 12, wherein said RAG-repressor is PTEN or SOCS3.

14. The method of claim 13, wherein said RAG-repressor is PTEN.

15. The method of claim 13, wherein said RAG-repressor is SOCS3.

16. The method of claim 12, wherein said compound is luteolin, quercetin, genistein, or phentolamine.

17. A method of increasing neuronal growth in a subject in need thereof, said method comprising administering to said subject an effective amount of a compound that inhibits expression or activity of a RAG-repressor, wherein said inhibition of said RAG-repressor increases expression of a regeneration-associated marker gene (RAG).

18. The method of claim 17, wherein said compound is ambroxol, luteolin, quercetin, genistein, phentolamine, felodipine, ticlodipine, sulconazole, propofol, isoconazole, azacyclonol, prenylamine, fendiline, hexetidine, cloperastine, drofenine, dienestrol, clioquinol, ivermectin, clorgiline, naftifine, quinisocaine, mefloquine, miconazole, clomifene, pxybutynin, loperamide, butoconazole, profenamine, vanoxerine, famprofazone, chlorhexidine, bromperidol, or amoxapine.

19. The method of claim 17, wherein said compound is luteolin, quercetin, genistein, or phentolamine.

20. The method of claim 12, where said RAG-repressor is PTEN or SOCS3.

21. The method of claim 12, wherein said RAG-repressor is PTEN.

22. The method of claim 12, wherein said RAG-repressor is SOCS3.

23. The method of claim 1, wherein said neuronal growth is neuronal regeneration.

24. The method of claim 23, wherein said neuronal regeneration comprises accelerating or improving neural repair in the CNS of said subject.

25. The method of claim 24, wherein said subject has experienced a traumatic injury to the CNS.

26. The method of claim 23, wherein said neuronal regeneration comprises accelerating or improving neural repair in the PNS of said subject.

27. The method of claim 23, wherein said neuronal regeneration comprises restoring neuronal function in said subject.

28. The method of claim 1, wherein said subject has a neurodegenerative disease.


SEQUENCE LISTING (68 sequences)

SEQ ID NO: 1 - DNA, 20 bp, Artificial Sequence
  Synthetic polynucleotide
ccacgctacttcctcctcaa

SEQ ID NO: 2 - DNA, 20 bp, Artificial Sequence
  Synthetic polynucleotide
cagcggatgatgaaggtgtt

SEQ ID NO: 3 - DNA, 20 bp, Artificial Sequence
  Synthetic polynucleotide
ccaggtctctgcctcagaag

SEQ ID NO: 4 - DNA, 20 bp, Artificial Sequence
  Synthetic polynucleotide
catctccaggggtctgttgt

SEQ ID NO: 5 - DNA, 21 bp, Artificial Sequence
  Synthetic polynucleotide
aagatcacagcaaaaggagca

SEQ ID NO: 6 - DNA, 20 bp, Artificial Sequence
  Synthetic polynucleotide
gagtttcggctttccagtca

SEQ ID NO: 7 - DNA, 20 bp, Artificial Sequence
  Synthetic polynucleotide
ttgatactgcagggcaagag

SEQ ID NO: 8 - DNA, 20 bp, Artificial Sequence
  Synthetic polynucleotide
tgagttatctcaggcaccca

SEQ ID NO: 9 - DNA, 20 bp, Artificial Sequence
  Synthetic polynucleotide
atttttcctgagccccaaag

SEQ ID NO: 10 - DNA, 20 bp, Artificial Sequence
  Synthetic polynucleotide
ctgggtacggttctccagac

SEQ ID NO: 11 - DNA, 20 bp, Artificial Sequence
  Synthetic polynucleotide
ccatgatgttctcgggtttc

SEQ ID NO: 12 - DNA, 20 bp, Artificial Sequence
  Synthetic polynucleotide
tgtcaccgtggggataaagt

SEQ ID NO: 13 - DNA, 20 bp, Artificial Sequence
  Synthetic polynucleotide
tcccagatccagatcaaacc

SEQ ID NO: 14 - DNA, 20 bp, Artificial Sequence
  Synthetic polynucleotide
gggtacctttcttgctgctt

SEQ ID NO: 15 - DNA, 20 bp, Artificial Sequence
  Synthetic polynucleotide
tgtttggcagcaagtaccag

SEQ ID NO: 16 - DNA, 20 bp, Artificial Sequence
  Synthetic polynucleotide
gctgtggagtcttccctgag

SEQ ID NO: 17 - DNA, 20 bp, Artificial Sequence
  Synthetic polynucleotide
caggaaagatcccaagtcca

SEQ ID NO: 18 - DNA, 20 bp, Artificial Sequence
  Synthetic polynucleotide
gaacggaacattgcacacac

SEQ ID NO: 19 - DNA, 20 bp, Artificial Sequence
  Synthetic polynucleotide
ttgtgtccctcgtgatgttt

SEQ ID NO: 20 - DNA, 20 bp, Artificial Sequence
  Synthetic polynucleotide
ccttaatcaagtccggcagt

SEQ ID NO: 21 - DNA, 20 bp, Artificial Sequence
  Synthetic polynucleotide
catgtatgccatcctcaacg

SEQ ID NO: 22 - DNA, 20 bp, Artificial Sequence
  Synthetic polynucleotide
ccacgtccacaaactcattg

SEQ ID NO: 23 - DNA, 20 bp, Artificial Sequence
  Synthetic polynucleotide
gaagcagcaagcatgatgag

SEQ ID NO: 24 - DNA, 20 bp, Artificial Sequence
  Synthetic polynucleotide
ggccactccaagtttccata

SEQ ID NO: 25 - DNA, 20 bp, Artificial Sequence
  Synthetic polynucleotide
acatgctcagggaacaggtg

SEQ ID NO: 26 - DNA, 21 bp, Artificial Sequence
  Synthetic polynucleotide
tcaaaacgtttgcaactgctg

SEQ ID NO: 27 - DNA, 20 bp, Artificial Sequence
  Synthetic polynucleotide
gtgccccaagattaagcaag

SEQ ID NO: 28 - DNA, 20 bp, Artificial Sequence
  Synthetic polynucleotide
gtgacagtccctgctgttca

SEQ ID NO: 29 - DNA, 20 bp, Artificial Sequence
  Synthetic polynucleotide
tgcgcttcagacatgagaac

SEQ ID NO: 30 - DNA, 21 bp, Artificial Sequence
  Synthetic polynucleotide
tctgtctccatgaggtcctgt

SEQ ID NO: 31 - DNA, 19 bp, Artificial Sequence
  Synthetic polynucleotide
gagggccaagttggacagt

SEQ ID NO: 32 - DNA, 20 bp, Artificial Sequence
  Synthetic polynucleotide
gccttttcgttgttttccaa

SEQ ID NO: 33 - DNA, 20 bp, Artificial Sequence
  Synthetic polynucleotide
ctctggtgaagctggaggac

SEQ ID NO: 34 - DNA, 20 bp, Artificial Sequence
  Synthetic polynucleotide
gcacctgttccttggaatgt

SEQ ID NO: 35 - DNA, 20 bp, Artificial Sequence
  Synthetic polynucleotide
aagatccacacaggcgagag

SEQ ID NO: 36 - DNA, 20 bp, Artificial Sequence
  Synthetic polynucleotide
aagctctggccacactctgt

SEQ ID NO: 37 - DNA, 20 bp, Artificial Sequence
  Synthetic polynucleotide
tctaatctcacccagcacca

SEQ ID NO: 38 - DNA, 20 bp, Artificial Sequence
  Synthetic polynucleotide
tagggcttctcgccagtatg

SEQ ID NO: 39 - DNA, 20 bp, Artificial Sequence
  Synthetic polynucleotide
caaaagaaccaagcccaatg

SEQ ID NO: 40 - DNA, 20 bp, Artificial Sequence
  Synthetic polynucleotide
tgctgggttagtcctgctct

SEQ ID NO: 41 - DNA, 20 bp, Artificial Sequence
  Synthetic polynucleotide
gtctggtgggtcagggacta

SEQ ID NO: 42 - DNA, 20 bp, Artificial Sequence
  Synthetic polynucleotide
ggtgaagatctcccagagca

SEQ ID NO: 43 - DNA, 20 bp, Artificial Sequence
  Synthetic polynucleotide
cacagcaggtttccatagca

SEQ ID NO: 44 - DNA, 20 bp, Artificial Sequence
  Synthetic polynucleotide
cccagcacatctaagcctgt

SEQ ID NO: 45 - DNA, 20 bp, Artificial Sequence
  Synthetic polynucleotide
attagccagcgaatccagac

SEQ ID NO: 46 - DNA, 20 bp, Artificial Sequence
  Synthetic polynucleotide
atcttgagctcggcagtgtt

SEQ ID NO: 47 - DNA, 20 bp, Artificial Sequence
  Synthetic polynucleotide
ctgtttgcctactcggacct

SEQ ID NO: 48 - DNA, 20 bp, Artificial Sequence
  Synthetic polynucleotide
cttcttctgctcctgcgagt

SEQ ID NO: 49 - DNA, 20 bp, Artificial Sequence
  Synthetic polynucleotide
attctggggccttttaatcc

SEQ ID NO: 50 - DNA, 20 bp, Artificial Sequence
  Synthetic polynucleotide
caatgaagcctcttgccaat

SEQ ID NO: 51 - DNA, 20 bp, Artificial Sequence
  Synthetic polynucleotide
gccattttcatcgctcaagt

SEQ ID NO: 52 - DNA, 20 bp, Artificial Sequence
  Synthetic polynucleotide
cagaaaccagtccactgcaa

SEQ ID NO: 53 - DNA, 20 bp, Artificial Sequence
  Synthetic polynucleotide
aggcacagcgagtacaatcc

SEQ ID NO: 54 - DNA, 20 bp, Artificial Sequence
  Synthetic polynucleotide
gagtgagggtaggtgctgct

SEQ ID NO: 55 - DNA, 20 bp, Artificial Sequence
  Synthetic polynucleotide
gcgctcattgcagttgttat

SEQ ID NO: 56 - DNA, 20 bp, Artificial Sequence
  Synthetic polynucleotide
tctgcaggctcataggtgac

SEQ ID NO: 57 - DNA, 20 bp, Artificial Sequence
  Synthetic polynucleotide
aagcccagacaatcaccttg

SEQ ID NO: 58 - DNA, 20 bp, Artificial Sequence
  Synthetic polynucleotide
gcacctggatccctgaagta

SEQ ID NO: 59 - DNA, 20 bp, Artificial Sequence
  Synthetic polynucleotide
ccccatggaaatcagacagt

SEQ ID NO: 60 - DNA, 20 bp, Artificial Sequence
  Synthetic polynucleotide
tcctggagattacgcttgct

SEQ ID NO: 61 - DNA, 20 bp, Artificial Sequence
  Synthetic polynucleotide
ggagcagagatgtgggaatg

SEQ ID NO: 62 - DNA, 20 bp, Artificial Sequence
  Synthetic polynucleotide
tggcaaggagtgggtctcta

SEQ ID NO: 63 - DNA, 20 bp, Artificial Sequence
  Synthetic polynucleotide
caccgctgctactgctactg

SEQ ID NO: 64 - DNA, 20 bp, Artificial Sequence
  Synthetic polynucleotide
gcaggcacttggaagttagc

SEQ ID NO: 65 - DNA, 20 bp, Artificial Sequence
  Synthetic polynucleotide
gcctgaagacacggactttc

SEQ ID NO: 66 - DNA, 20 bp, Artificial Sequence
  Synthetic polynucleotide
cagcactggaccatgacatt

SEQ ID NO: 67 - DNA, 20 bp, Artificial Sequence
  Synthetic polynucleotide
gctaacggggacttcttcaa

SEQ ID NO: 68 - DNA, 20 bp, Artificial Sequence
  Synthetic polynucleotide
tgtgttatctgccacctcca